(19) United States
(12) Patent Application Publication
(10) Pub. No.: US 2024/0409273 A1
(43) Pub. Date: Dec. 12, 2024

(54) Reusable Packaging System, Apparatus, and Methods for Textile Items

(71) Applicant: REVOLUTION-ZERO GROUP LTD., Alton (GB)

(72) Inventors: Thomas Andrew Dawson, Alton (GB); Benjamin Ridgewell-Manson, Crediton (GB)

(21) Appl. No.: 18/738,098

(22) Filed: Jun. 10, 2024

Related U.S. Application Data
(60) Provisional application No. 63/472,295, filed on Jun. 11, 2023.

(51) Int. Cl. B65D 30/00; B65D 33/16; D06F 95/00

(52) U.S. Cl. CPC B65D 29/00; B65D 33/1608; D06F 95/006

(57) ABSTRACT

A package comprises a sterilized, reusable textile item comprising a textile material; a reusable bag comprising a sterilized textile material defining an interior cargo space in which the sterilized textile item is contained; a reusable envelope comprising a folded, sheet of sterilized textile material by which the bag containing the sterilized textile item is entirely enveloped; and a reusable tie comprising an elongate length of a sterilized textile material extending around and securing the envelope onto the bag. A method comprises inserting the textile item into the bag; folding the sheet about the bag; and securing the folded sheet enveloping the bag using the tie. All four components are reusable of the packaging including the textile item. The package preferably constitutes mono material packaging.

### COPYRIGHT STATEMENT

Any new and original work of authorship in this document is subject to copyright protection under the copyright laws of the United States and other countries. Reproduction by anyone of this document as it appears in official governmental records is permitted, but otherwise all other copyright rights whatsoever are reserved.

### BACKGROUND OF THE INVENTION

The field of the invention relates to packaging systems for textiles that are used in medical and healthcare sectors, but embodiments in accordance with one or more aspects and features of the invention can be utilized for other sectors in which packaging of items are required to be kept in relatively protected and aseptic conditions.

Textiles for medical use are typically delivered in disposable packaging systems and are often double or triple packaged, not including cartons, to preserve the aseptic function and integrity of textiles. This is especially true of textiles such as surgical drapes and gowns which are required to be sterile for use in sterile, highly controlled operating theatre environments.

When a sterile item is removed from its package it needs to be done in a way that does not contaminate the item. Typically, an item is laid onto a disposable sterile covering without being touched by the operator.

All of these above processes require a considerable amount of material, all of which is single-use and goes into waste streams. From a general systems resource management perspective, this level of waste is unsustainable. A need exists for addressing this that is sustainable. One or more embodiments of the invention are believed to address such need.

### SUMMARY OF THE INVENTION

The invention includes many aspects and features.

In an aspect of the invention, a package comprises: (a) a sterilized, reusable textile item comprising a textile material; (b) a reusable bag comprising a sterilized textile material defining an interior cargo space in which the sterilized textile item is contained; (c) a reusable envelope comprising a folded, sheet of sterilized textile material by which the bag containing the sterilized textile item is entirely enveloped; and (d) a reusable tie comprising an elongate length of a sterilized textile material extending around and securing the envelope onto the bag. The package preferably constitutes mono material packaging.

In a feature, the sterilized, reusable textile item comprises a textile article used in foodservice. Examples of this include the textile article comprising an apron; and the textile article comprising a head covering.

In a feature, the sterilized, reusable textile item comprises a textile article used in healthcare. Examples of this include the textile article comprising a gown; the textile article comprising a surgical drape; the textile article comprising scrubs; the textile article comprising a sanitary piece of textile clothing worn by a healthcare provider; and the textile article comprising a sanitary piece of textile clothing worn by a healthcare patient.

In a feature, the bag further comprises fasteners configured to secure the bag in the closed state. In examples, the fasteners comprise a sterilized textile material, such as straps; and the fasteners comprise a sterilized non-textile material, such as buckles.

In a feature, the tie comprises a strap.

In a feature, the tie extends completely around the envelope. In examples of this, the tie extends completely around a lengthwise direction of the envelope; and the tie extends completely around a widthwise direction of the envelope.

In a feature, the tie comprises distal ends that are knotted together.

In a feature, the tie comprises distal ends that fasten together to secure the envelope onto the bag. The tie further may comprise fasteners at the distal ends by which the distal ends are fastened together to secure the envelope onto the bag.

In a feature, the textile material of the textile item comprises at least one of nylon, polyester, cotton, flax, and hemp.

In a feature, the textile material of the bag comprises at least one of nylon, polyester, cotton, flax, and hemp.

In a feature, the textile material of the sheet comprises at least one of nylon, polyester, cotton, flax, and hemp.

In a feature, the textile material of the tie comprises at least one of nylon, polyester, cotton, flax, and hemp.

In a feature, the textile material of the bag, the textile material of the sheet, and the textile material of the tie are the same textile material.

In a feature, the textile material of the textile item, the textile material of the bag, the textile material of the sheet, and the textile material of the tie are the same textile material.

In a feature, the textile material of the textile item, the textile material of the bag, the textile material of the sheet, and the textile material of the tie are repellant, semi-impermeable, or impermeable to water.

In a feature, the textile material of the textile item, the textile material of the bag, the textile material of the sheet, and the textile material of the tie are coated with a hydrophobic substance.

In a feature, surfaces of the textile item, the bag, the sheet, and the tie each comprise a coating of a member of the C6 fluorocarbon family or C6 fluorotelomers.

In another aspect, a method comprises: (a) inserting a sterilized textile item through an open end of a bag into an interior space of the bag and closing the open end of the bag such that the bag completely contains the item within the interior space, the bag comprising a sterilized textile material defining the interior space of the bag; (b) folding a sheet of a sterilized textile material about the bag into a configuration such that the folded sheet of textile material defines an envelope that entirely envelops the bag; and (c) securing the folded sheet of material in the configuration enveloping of the bag using an elongate length of a sterilized textile material. The resulting package preferably constitutes mono material packaging.

In a feature, the method further comprises, before said steps (a), (b), and (c), sterilizing the textile item, sterilizing the sheet to be folded to define the envelope, and sterilizing the tie by washing, heating, or washing and heating. The sterilizing of the textile item, the sterilizing of the sheet, and the sterilizing of the tie are performed in the same process. Alternatively, the sterilizing of the textile item, the sterilizing of the sheet, and the sterilizing of the tie are performed in different processes.

In a feature, the method further comprises, after said steps (a), (b), and (c), and after the textile item has been unpacked and used, sterilizing the textile item, sterilizing the sheet to be folded to define the envelope, and sterilizing the tie by washing, heating, or washing and heating. The sterilizing of the textile item, the sterilizing of the sheet, and the sterilizing of the tie are performed in the same process. Alternatively, the sterilizing of the textile item, the sterilizing of the sheet, and the sterilizing of the tie are performed in different processes.

In a feature, the method further comprises repackaging the textile item. Repackaging comprises comprising: inserting the textile item through an open end of a bag into an interior space of the bag and closing the open end of the bag such that the bag completely contains the item within the interior space, the bag comprising a textile material; folding a sheet of a textile material about the bag into a configuration such that the folded sheet of textile material defines an envelope that entirely envelops the bag; and securing the folded sheet of material in the configuration enveloping of the bag. The bag, sheet, and/or the tie may be the same or different from the prior packaging of the textile item.

In a feature, the sterilized, reusable textile item comprises a textile article used in foodservice. Examples include the textile article comprising an apron; and the textile article comprising a head covering.

In a feature, the sterilized, reusable textile item comprises a textile article used in healthcare. Examples include the textile article comprising a gown; the textile article comprising scrubs; the textile article comprising a sanitary piece of textile clothing worn by a healthcare provider; and the textile article comprising a sanitary piece of textile clothing worn by a healthcare patient.

Additional aspects and features are disclosed in the appendices to the specification, which is incorporated herein by reference, and in the following detailed descriptions and accompanying drawings.

In addition to the aforementioned aspects and features of the invention, it should be noted that the invention further encompasses the various logical combinations and subcombinations of such aspects and features. Thus, for example, claims in this or a divisional or continuing patent application or applications may be separately directed to any aspect, feature, or embodiment disclosed herein, or combination thereof, without requiring any other aspect, feature, or embodiment.

### BRIEF DESCRIPTION OF THE DRAWINGS

One or more preferred embodiments of the invention now will be described in detail with reference to the accompanying drawings, wherein the same elements are referred to with the same reference numerals.

FIG. 1 is a front perspective view of a textile item being inserted into a bag of a preferred embodiment of a method in accordance with one or more aspects and features of the invention.

FIG. 2 is a front perspective view of the bag containing the textile item of FIG. 1.

FIG. 3 is a front perspective view of the bag in a closed configuration completely containing the textile item within the bag.

FIG. 4 is a front perspective view of the bag secured in the closed configuration with the textile item completely contained within the bag.

FIG. 5 is a front perspective view of a textile item being inserted into a bag of another preferred embodiment of a method in accordance with one or more aspects and features of the invention.

FIG. 6 is a front perspective view of the bag containing the textile item of FIG. 1.

FIG. 7 is a front perspective view of the bag in a closed configuration completely containing the textile item within the bag.

FIG. 8 is a front perspective view of the bag secured in the closed configuration with the textile item completely contained within the bag.

FIG. 9 is a front perspective view of a textile item being inserted into a bag of another preferred embodiment of a method in accordance with one or more aspects and features of the invention.

FIG. 10 is a front perspective view of the bag containing the textile item of FIG. 1.

FIG. 11 is a front perspective view of the bag in a closed configuration completely containing the textile item within the bag.

FIG. 12 is a front perspective view of the bag secured in the closed configuration with the textile item completely contained within the bag.

FIG. 13 is a front perspective view of a textile item being inserted into a bag of another preferred embodiment of a method in accordance with one or more aspects and features of the invention.

FIG. 14 is a front perspective view of the bag containing the textile item of FIG. 1.

FIG. 15 is a front perspective view of the bag in a closed configuration completely containing the textile item within the bag.

FIG. 16 is a front perspective view of the bag secured in the closed configuration with the textile item completely contained within the bag.

FIG. 17 is a front perspective view of a textile item being inserted into a bag of another preferred embodiment of a method in accordance with one or more aspects and features of the invention.

FIG. 18 is a front perspective view of the bag containing the textile item of FIG. 1.

FIG. 19 is a front perspective view of the bag in a closed configuration completely containing the textile item within the bag.

FIG. 20 is a front perspective view of the bag secured in the closed configuration with the textile item completely contained within the bag.

FIG. 21 is a front perspective view of a textile item being inserted into a bag of another preferred embodiment of a method in accordance with one or more aspects and features of the invention.

FIG. 22 is a front perspective view of the bag containing the textile item of FIG. 1.

FIG. 23 is a front perspective view of the bag in a closed configuration completely containing the textile item within the bag.

FIG. 24 is a front perspective view of the bag secured in the closed configuration with the textile item completely contained within the bag.

FIG. 25 is a front perspective view of a textile item being inserted into a bag of another preferred embodiment of a method in accordance with one or more aspects and features of the invention.

FIG. 26 is a front perspective view of the bag containing the textile item of FIG. 1.

FIG. 27 is a front perspective view of the bag in a closed configuration completely containing the textile item within the bag.

FIG. 28 is a front perspective view of the bag secured in the closed configuration with the textile item completely contained within the bag.

FIG. 29 is a front perspective view of a textile item being inserted into a bag of another preferred embodiment of a method in accordance with one or more aspects and features of the invention.

FIG. 30 is a front perspective view of the bag containing the textile item of FIG. 1.

FIG. 31 is a front perspective view of the bag in a closed configuration completely containing the textile item within the bag.

FIG. 32 is a front perspective view of the bag secured in the closed configuration with the textile item completely contained within the bag.

FIG. 33 is a front perspective view of a textile item being inserted into a bag of another preferred embodiment of a method in accordance with one or more aspects and features of the invention.

FIG. 34 is a front perspective view of the bag containing the textile item of FIG. 1.

FIG. 35 is a front perspective view of the bag in a closed configuration completely containing the textile item within the bag.

FIG. 36 is a front perspective view of the bag secured in the closed configuration with the textile item completely contained within the bag.

FIG. 37 is a front perspective view of a textile item being inserted into a bag of another preferred embodiment of a method in accordance with one or more aspects and features of the invention.

FIG. 38 is a front perspective view of the bag containing the textile item of FIG. 1.

FIG. 39 is a front perspective view of the bag in a closed configuration completely containing the textile item within the bag.

FIG. 40 is a front perspective view of the bag secured in the closed configuration with the textile item completely contained within the bag.

FIG. 41 is a front perspective view of a textile item being inserted into a bag of another preferred embodiment of a method in accordance with one or more aspects and features of the invention.

FIG. 42 is a front perspective view of the bag containing the textile item of FIG. 1.

FIG. 43 is a front perspective view of the bag in a closed configuration completely containing the textile item within the bag.

FIG. 44 is a front perspective view of the bag secured in the closed configuration with the textile item completely contained within the bag.

FIG. 45 is a front perspective view of a textile item being inserted into a bag of another preferred embodiment of a method in accordance with one or more aspects and features of the invention.

FIG. 46 is a front perspective view of the bag containing the textile item of FIG. 1.

FIG. 47 is a front perspective view of the bag in a closed configuration completely containing the textile item within the bag.

FIG. 48 is a front perspective view of the bag secured in the closed configuration with the textile item completely contained within the bag.

FIG. 49 is a front perspective view of a textile item being inserted into a bag of another preferred embodiment of a method in accordance with one or more aspects and features of the invention.

FIG. 50 is a front perspective view of the bag containing the textile item of FIG. 1.

FIG. 51 is a front perspective view of the bag in a closed configuration completely containing the textile item within the bag.

FIG. 52 is a front perspective view of the bag secured in the closed configuration with the textile item completely contained within the bag.

FIG. 53 is a front perspective view of a textile item being inserted into a bag of another preferred embodiment of a method in accordance with one or more aspects and features of the invention.

FIG. 54 is a front perspective view of the bag containing the textile item of FIG. 1.

FIG. 55 is a front perspective view of the bag in a closed configuration completely containing the textile item within the bag.

FIG. 56 is a front perspective view of the bag secured in the closed configuration with the textile item completely contained within the bag.

FIG. 57 is a front elevational view of the bag of FIG. 1.

FIG. 58 is a perspective view of the bag of FIG. 57.

FIG. 59 is a top plan view of the bag of FIG. 57.

FIG. 60 is a side plan view of the bag of FIG. 57.

FIG. 61 is a front elevational view of the bag of FIG. 57 secured in the closed configuration.

FIG. 62 is a front elevational view of another bag of a preferred embodiment of a method in accordance with one or more aspects and features of the invention.

FIG. 63 is a perspective view of the bag of FIG. 62.

FIG. 64 is a top plan view of the bag of FIG. 62.

FIG. 65 is a side plan view of the bag of FIG. 62.

FIG. 66 is a front elevational view of the bag of FIG. 62 secured in the closed configuration.

FIG. 67 is a front elevational view of the bag of FIG. 13.

FIG. 68 is a perspective view of the bag of FIG. 67.

FIG. 69 is a top plan view of the bag of FIG. 67.

FIG. 70 is a side plan view of the bag of FIG. 67.

FIG. 71 is a front elevational view of the bag of FIG. 67 secured in the closed configuration.

FIG. 72 is a front elevational view of the bag of FIG. 21.

FIG. 73 is a perspective view of the bag of FIG. 72.

FIG. 74 is a top plan view of the bag of FIG. 72.

FIG. 75 is a side plan view of the bag of FIG. 72.

FIG. 76 is a front elevational view of the bag of FIG. 72 secured in the closed configuration.

FIG. 77 is a front elevational view of another bag of a preferred embodiment of a method in accordance with one or more aspects and features of the invention.

FIG. 78 is a perspective view of the bag of FIG. 77.

FIG. 79 is a top plan view of the bag of FIG. 77.

FIG. 80 is a side plan view of the bag of FIG. 77.

FIG. 81 is a front elevational view of the bag of FIG. 77 secured in the closed configuration.

FIG. 82 is a front elevational view of the bag of FIG. 29.

FIG. 83 is a perspective view of the bag of FIG. 82.

FIG. 84 is a top plan view of the bag of FIG. 82.

FIG. 85 is a side plan view of the bag of FIG. 82.

FIG. 86 is a front elevational view of the bag of FIG. 82 secured in the closed configuration.

FIG. 87 is a front elevational view of the bag of FIG. 33.

FIG. 88 is a perspective view of the bag of FIG. 87.

FIG. 89 is a top plan view of the bag of FIG. 87.

FIG. 90 is a side plan view of the bag of FIG. 87.

FIG. 91 is a front elevational view of the bag of FIG. 87 secured in the closed configuration.

FIG. 92 is a front elevational view of the bag of FIG. 37.

FIG. 93 is a perspective view of the bag of FIG. 92.

FIG. 94 is a top plan view of the bag of FIG. 92.

FIG. 95 is a side plan view of the bag of FIG. 92.

FIG. 96 is a front elevational view of the bag of FIG. 92 secured in the closed configuration.

FIG. 97 is a front elevational view of the bag of FIG. 41.

FIG. 98 is a perspective view of the bag of FIG. 97.

FIG. 99 is a top plan view of the bag of FIG. 97.

FIG. 100 is a side plan view of the bag of FIG. 97.

FIG. 101 is a front elevational view of the bag of FIG. 97 secured in the closed configuration.

FIG. 102 is a front elevational view of the bag of FIG. 45.

FIG. 103 is a perspective view of the bag of FIG. 102.

FIG. 104 is a top plan view of the bag of FIG. 102.

FIG. 105 is a side plan view of the bag of FIG. 102.

FIG. 106 is a front elevational view of the bag of FIG. 102 secured in the closed configuration.

FIG. 107 is a front elevational view of the bag of FIG. 49.

FIG. 108 is a perspective view of the bag of FIG. 107.

FIG. 109 is a top plan view of the bag of FIG. 107.

FIG. 110 is a side plan view of the bag of FIG. 107.

FIG. 111 is a front elevational view of the bag of FIG. 107 secured in the closed configuration.

FIG. 112 is a front elevational view of the bag of FIG. 53.

FIG. 113 is a perspective view of the bag of FIG. 112.

FIG. 114 is a top plan view of the bag of FIG. 112.

FIG. 115 is a side plan view of the bag of FIG. 112.

FIG. 116 is a front elevational view of the bag of FIG. 112 secured in the closed configuration.

FIG. 117 is an enlarged view of FIG. 4.

FIGS. 118-122 illustrate a method of creating an envelope encompassing a bag that has been secured in a closed configuration by enveloping the bag within a sheet by folding, in accordance with one or more aspects and features of the present invention.

FIGS. 123-128 illustrate steps of another preferred method of enveloping a bag secured in a closed configuration by folding a sheet around the bag to entirely envelope the bag, in accordance with one or more aspects and features of the invention.

FIGS. 129-133 illustrate steps of a method of enveloping a bag secured in a closed configuration by folding a sheet around the bag to entirely envelope the bag, in accordance with one or more aspects and features of the invention.

FIG. 134 is a top plan view of a preferred tie that may be used to secure the envelope of FIG. 133, in accordance with one or more aspects and features of the invention.

FIG. 135 is a top plan view of the tie of FIG. 134 used in a first preferred manner to secure the envelope of FIG. 133, in accordance with one or more aspects and features of the invention.

FIG. 136 is a top plan view of the tie of FIG. 134 used in a second preferred manner to secure the envelope of FIG. 133, in accordance with one or more aspects and features of the invention.

FIG. 137 is a top plan view of the tie of FIG. 134 used in a third preferred manner to secure the envelope of FIG. 133, in accordance with one or more aspects and features of the invention.

FIG. 138 is a top plan view of another preferred tie that may be used to secure the envelope of FIG. 133, in accordance with one or more aspects and features of the invention.

FIG. 139 is a top plan view of the tie of FIG. 138 used in a first preferred manner to secure the envelope of FIG. 133, in accordance with one or more aspects and features of the invention.

FIG. 140 is a top plan view of the tie of FIG. 138 used in a second preferred manner to secure the envelope of FIG. 133, in accordance with one or more aspects and features of the invention.

FIG. 141 is a top plan view of two ties of FIG. 138 used in a preferred manner to secure the envelope of FIG. 133, in accordance with one or more aspects and features of the invention.

FIG. 142 is a top plan view of another preferred tie that may be used to secure the envelope of FIG. 133, in accordance with one or more aspects and features of the invention.

FIG. 143 is a top plan view of the tie of FIG. 142 used in a first preferred manner to secure the envelope of FIG. 133, in accordance with one or more aspects and features of the invention.

FIG. 144 is a top plan view of the tie of FIG. 142 used in a second preferred manner to secure the envelope of FIG. 133, in accordance with one or more aspects and features of the invention.

FIG. 145 is a top plan view of two ties of FIG. 142 used in a preferred manner to secure the envelope of FIG. 133, in accordance with one or more aspects and features of the invention.

FIG. 146 is a top plan view of another preferred tie that may be used to secure the envelope of FIG. 133, in accordance with one or more aspects and features of the invention.

FIG. 147 is a top plan view of the tie of FIG. 146 used in a first preferred manner to secure the envelope of FIG. 133, in accordance with one or more aspects and features of the invention.

FIG. 148 is a top plan view of the tie of FIG. 146 used in a second preferred manner to secure the envelope of FIG. 133, in accordance with one or more aspects and features of the invention.

FIG. 149 is a top plan view of two ties of FIG. 146 used in a preferred manner to secure the envelope of FIG. 133, in accordance with one or more aspects and features of the invention.

FIG. 150 is a top plan view of another preferred tie that may be used to secure the envelope of FIG. 133, in accordance with one or more aspects and features of the invention.

FIG. 151 is a top plan view of the tie of FIG. 150 used in a first preferred manner to secure the envelope of FIG. 133, in accordance with one or more aspects and features of the invention.

FIG. 152 is a top plan view of the tie of FIG. 150 used in a second preferred manner to secure the envelope of FIG. 133, in accordance with one or more aspects and features of the invention.

FIG. 153 is a top plan view of two ties of FIG. 150 used in a preferred manner to secure the envelope of FIG. 133, in accordance with one or more aspects and features of the invention.

FIG. 154 is a top plan view of another preferred tie that may be used to secure the envelope of FIG. 133, in accordance with one or more aspects and features of the invention.

FIG. 155 is a top plan view of the tie of FIG. 154 used in a first preferred manner to secure the envelope of FIG. 133, in accordance with one or more aspects and features of the invention.

FIG. 156 is a top plan view of the tie of FIG. 154 used in a second preferred manner to secure the envelope of FIG. 133, in accordance with one or more aspects and features of the invention.

FIG. 157 is a top plan view of two ties of FIG. 154 used in a preferred manner to secure the envelope of FIG. 133, in accordance with one or more aspects and features of the invention.

FIG. 158 is a top plan view of another preferred tie that may be used to secure the envelope of FIG. 133, in accordance with one or more aspects and features of the invention.

FIG. 159 is a top plan view of the tie of FIG. 158 used in a first preferred manner to secure the envelope of FIG. 133, in accordance with one or more aspects and features of the invention.

FIG. 160 is a top plan view of the tie of FIG. 158 used in a second preferred manner to secure the envelope of FIG. 133, in accordance with one or more aspects and features of the invention.

FIG. 161 is a top plan view of two ties of FIG. 158 used in a preferred manner to secure the envelope of FIG. 133, in accordance with one or more aspects and features of the invention.

FIG. 162 is a top plan view of another preferred tie that may be used to secure the envelope of FIG. 133, in accordance with one or more aspects and features of the invention.

FIG. 163 is a top plan view of the tie of FIG. 162 used in a first preferred manner to secure the envelope of FIG. 133, in accordance with one or more aspects and features of the invention.

FIG. 164 is a top plan view of the tie of FIG. 162 used in a second preferred manner to secure the envelope of FIG. 133, in accordance with one or more aspects and features of the invention.

FIG. 165 is a top plan view of two ties of FIG. 162 used in a preferred manner to secure the envelope of FIG. 133, in accordance with one or more aspects and features of the invention.

FIG. 166 is a top plan view of another preferred tie that may be used to secure the envelope of FIG. 133, in accordance with one or more aspects and features of the invention.

FIG. 167 is a top plan view of the tie of FIG. 166 used in a first preferred manner to secure the envelope of FIG. 133, in accordance with one or more aspects and features of the invention.

FIG. 168 is a top plan view of the tie of FIG. 166 used in a second preferred manner to secure the envelope of FIG. 133, in accordance with one or more aspects and features of the invention.

FIG. 169 is a top plan view of two ties of FIG. 166 used in a preferred manner to secure the envelope of FIG. 133, in accordance with one or more aspects and features of the invention.

FIG. 170 is a top plan view of another preferred tie that may be used to secure the envelope of FIG. 133, in accordance with one or more aspects and features of the invention.

FIG. 171 is a top plan view of the tie of FIG. 170 used in a first preferred manner to secure the envelope of FIG. 133, in accordance with one or more aspects and features of the invention.

FIG. 172 is a top plan view of the tie of FIG. 170 used in a second preferred manner to secure the envelope of FIG. 133, in accordance with one or more aspects and features of the invention.

FIG. 173 is a top plan view of two ties of FIG. 170 used in a preferred manner to secure the envelope of FIG. 133, in accordance with one or more aspects and features of the invention.

FIG. 174 is a top plan view of another preferred tie that may be used to secure the envelope of FIG. 133, in accordance with one or more aspects and features of the invention.

FIG. 175 is a top plan view of the tie of FIG. 174 used in a first preferred manner to secure the envelope of FIG. 133, in accordance with one or more aspects and features of the invention.

FIG. 176 is a top plan view of the tie of FIG. 174 used in a second preferred manner to secure the envelope of FIG. 133, in accordance with one or more aspects and features of the invention.

FIG. 177 is a top plan view of two ties of FIG. 174 used in a preferred manner to secure the envelope of FIG. 133, in accordance with one or more aspects and features of the invention.

FIG. 178 is a top plan view of another preferred tie that may be used to secure the envelope of FIG. 133, in accordance with one or more aspects and features of the invention.

FIG. 179 is a top plan view of the tie of FIG. 178 used in a first preferred manner to secure the envelope of FIG. 133, in accordance with one or more aspects and features of the invention.

FIG. 180 is a top plan view of the tie of FIG. 178 used in a second preferred manner to secure the envelope of FIG. 133, in accordance with one or more aspects and features of the invention.

FIG. 181 is a top plan view of two ties of FIG. 178 used in a preferred manner to secure the envelope of FIG. 133, in accordance with one or more aspects and features of the invention.

FIG. 182 is a top plan view of another preferred tie that may be used to secure the envelope of FIG. 133, in accordance with one or more aspects and features of the invention.

FIG. 183 is a top plan view of the tie of FIG. 182 used in a first preferred manner to secure the envelope of FIG. 133, in accordance with one or more aspects and features of the invention.

FIG. 184 is a top plan view of the tie of FIG. 182 used in a second preferred manner to secure the envelope of FIG. 133, in accordance with one or more aspects and features of the invention.

FIG. 185 is a top plan view of two ties of FIG. 182 used in a preferred manner to secure the envelope of FIG. 133, in accordance with one or more aspects and features of the invention.

FIG. 186 is a top plan view of another preferred tie that may be used to secure the envelope of FIG. 133, in accordance with one or more aspects and features of the invention.

FIG. 187 is a top plan view of the tie of FIG. 186 used in a first preferred manner to secure the envelope of FIG. 133, in accordance with one or more aspects and features of the invention.

FIG. 188 is a top plan view of the tie of FIG. 186 used in a second preferred manner to secure the envelope of FIG. 133, in accordance with one or more aspects and features of the invention.

FIG. 189 is a top plan view of two ties of FIG. 186 used in a preferred manner to secure the envelope of FIG. 133, in accordance with one or more aspects and features of the invention.

FIG. 190 is a top plan view of another preferred tie that may be used to secure the envelope of FIG. 133, in accordance with one or more aspects and features of the invention.

FIG. 191 is a top plan view of the tie of FIG. 190 used in a first preferred manner to secure the envelope of FIG. 133, in accordance with one or more aspects and features of the invention.

FIG. 192 is a top plan view of the tie of FIG. 190 used in a second preferred manner to secure the envelope of FIG. 133, in accordance with one or more aspects and features of the invention.

FIG. 193 is a top plan view of two ties of FIG. 190 used in a preferred manner to secure the envelope of FIG. 133, in accordance with one or more aspects and features of the invention.

FIG. 194 is a top plan view of another preferred tie that may be used to secure the envelope of FIG. 133, in accordance with one or more aspects and features of the invention.

FIG. 195 is a top plan view of the tie of FIG. 194 used in a first preferred manner to secure the envelope of FIG. 133, in accordance with one or more aspects and features of the invention.

FIG. 196 is a top plan view of the tie of FIG. 194 used in a second preferred manner to secure the envelope of FIG. 133, in accordance with one or more aspects and features of the invention.

FIG. 197 is a top plan view of two ties of FIG. 194 used in a preferred manner to secure the envelope of FIG. 133, in accordance with one or more aspects and features of the invention.

FIG. 198 is a front elevational view of another bag of a preferred embodiment of a method in accordance with one or more aspects and features of the invention.

FIG. 199 is a perspective view of the bag of FIG. 198.

FIG. 200 is a top plan view of the bag of FIG. 198.

FIG. 201 is a side plan view of the bag of FIG. 198.

FIG. 202 is a front elevational view of the bag of FIG. 198 secured in the closed configuration.

FIG. 203 is a front elevational view of another preferred bag in accordance with one or more aspects and features of the invention.

FIG. 204 is a front elevational view of another preferred bag in accordance with one or more aspects and features of the invention.

FIG. 205 is a front elevational view of another preferred bag in accordance with one or more aspects and features of the invention.

FIG. 206 is a front elevational view of the bag of FIG. 205 secured in the closed configuration.

FIG. 207 is a front elevational view of another preferred bag in accordance with one or more aspects and features of the invention.

FIG. 208 is a front elevational view of another preferred bag in accordance with one or more aspects and features of the invention.

FIG. 209 is a front elevational view of another bag of a preferred embodiment of a method in accordance with one or more aspects and features of the invention.

FIG. 210 is a perspective view of the bag of FIG. 209.

FIG. 211 is a top plan view of the bag of FIG. 209.

FIG. 212 is a side plan view of the bag of FIG. 209.

FIG. 213 is a front elevational view of the bag of FIG. 209 secured in the closed configuration.

FIG. 214 is a front elevational view of another bag of a preferred embodiment of a method in accordance with one or more aspects and features of the invention.

FIG. 215 is a perspective view of the bag of FIG. 214.

FIG. 216 is a top plan view of the bag of FIG. 214.

FIG. 217 is a side plan view of the bag of FIG. 214.

FIG. 218 is a front elevational view of the bag of FIG. 214 secured in the closed configuration.

FIG. 219 is a front elevational view of another bag of a preferred embodiment of a method in accordance with one or more aspects and features of the invention.

FIG. 220 is a perspective view of the bag of FIG. 219.

FIG. 221 is a top plan view of the bag of FIG. 219.

FIG. 222 is a side plan view of the bag of FIG. 219.

FIG. 223 is a front elevational view of the bag of FIG. 219 secured in the closed configuration.

FIGS. 224-226 illustrate preliminary steps of a preferred method of enveloping a bag secured in a closed configuration by folding a sheet around the bag to entirely envelope the bag, in accordance with one or more aspects and features of the invention.

FIG. 227 illustrates an alternative sheet of material used in enveloping a bag secured in a closed configuration, in accordance with one or more aspects and features of the invention.

FIG. 228 illustrates an alternative sheet of material used in enveloping a bag secured in a closed configuration, in accordance with one or more aspects and features of the invention.

FIG. 229 is a top plan view of another preferred embodiment of an envelope secured about a bag in accordance with one or more aspects and features of the invention.

FIG. 230 is a top plan view of another preferred embodiment of an envelope secured about a bag in accordance with one or more aspects and features of the invention.

FIG. 231 is a top plan view of another preferred embodiment of an envelope secured about a bag in accordance with one or more aspects and features of the invention.

FIG. 232 is a front elevational view of another preferred embodiment of a bag in accordance with one or more aspects and features of the invention.

FIG. 233 is a perspective view of the bag of FIG. 232.

FIGS. 234-238 illustrate steps of another preferred method of packaging a textile item using the bag of FIG. 232, in accordance with one or more aspects and features of the invention.

FIG. 239 illustrates a first sequence of illustrations 1-5 in which a textile item is inserted and secured within a bag,

FIG. 240 illustrates a second sequence of illustrations 1-6 subsequent to that of FIG. 239 in which the bag in turn is enveloped and secured within a sheet.

FIG. 241 illustrates part of the first sequence of FIG. 239.

FIG. 242 illustrates another part of the first sequence of FIG. 239.

FIG. 243 illustrates another part of the first sequence of FIG. 239.

FIG. 244 further illustrates the insertion of the textile item of FIG. 242 into the bag of FIG. 241.

FIG. 245 illustrates the textile item of FIG. 242 having been inserted into the bag of FIG. 241.

FIG. 246 illustrates further the textile item of FIG. 242 having been inserted into the bag of FIG. 241.

FIG. 247 illustrates another part of the first sequence of FIG. 239.

FIG. 248 illustrates another part of the first sequence of FIG. 239.

FIG. 249 illustrates another part of the first sequence of FIG. 239.

FIG. 250 illustrates part of the subsequent sequence of FIG. 240.

FIG. 251 illustrates another part of the subsequent sequence of FIG. 240.

FIG. 252 illustrates another part of the subsequent sequence of FIG. 240.

FIG. 253 illustrates another part of the subsequent sequence of FIG. 240.

FIG. 254 illustrates another part of the subsequent sequence of FIG. 240.

FIG. 255 illustrates another part of the subsequent sequence of FIG. 240.

FIG. 256 illustrates a final, optional step of the subsequent sequence of FIG. 240.

### DETAILED DESCRIPTION

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Furthermore, an embodiment of the invention may incorporate only one or a plurality of the aspects of the invention disclosed herein; only one or a plurality of the features disclosed herein; or combination thereof. As such, many embodiments are implicitly disclosed herein and fall within the scope of what is regarded as the invention.

Accordingly, while the invention is described herein in detail in relation to one or more embodiments, it is to be understood that this disclosure is illustrative and exemplary of the invention and is made merely for the purposes of providing a full and enabling disclosure of the invention. The detailed disclosure herein of one or more embodiments is not intended, nor is to be construed, to limit the scope of patent protection afforded the invention in any claim of a patent issuing here from, which scope is to be defined by the claims and the equivalents thereof. It is not intended that the scope of patent protection afforded the invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

With regard solely to construction of any claim with respect to the United States, no claim element is to be interpreted under 35 U.S.C. 112 (f) unless the explicit phrase “means for” or “step for” is actually used in such claim element, whereupon this statutory provision is intended to and should apply in the interpretation of such claim element. With regard to any method claim including a condition precedent step, such method requires the condition precedent to be met and the step to be performed at least once but not necessarily every time during performance of the claimed method.

Furthermore, it is important to note that, as used herein. “comprising” is open-ended insofar as that which follows such term is not exclusive. Additionally. “a” and “an” each generally denotes “at least one” but does not exclude a plurality unless the contextual use dictates otherwise. Thus, reference to “a picnic basket having an apple” is the same as “a picnic basket comprising an apple” and “a picnic basket including an apple”, each of which identically describes “a picnic basket having at least one apple” as well as “a picnic basket having apples”: the picnic basket further may contain one or more other items beside an apple. In contrast, reference to “a picnic basket having a single apple” describes “a picnic basket having only one apple”: the picnic basket further may contain one or more other items beside an apple. In contrast. “a picnic basket consisting of an apple” has only a single item contained therein, i.e., one apple; the picnic basket contains no other item.

When used herein to join a list of items. “or” denotes “at least one of the items” but does not exclude a plurality of items of the list. Thus, reference to “a picnic basket having cheese or crackers” describes “a picnic basket having cheese without crackers”. “a picnic basket having crackers without cheese”, and “a picnic basket having both cheese and crackers”: the picnic basket further may contain one or more other items beside cheese and crackers.

When used herein to join a list of items. “and” denotes “all of the items of the list”. Thus, reference to “a picnic basket having cheese and crackers” describes “a picnic basket having cheese, wherein the picnic basket further has crackers”, as well as describes “a picnic basket having crackers, wherein the picnic basket further has cheese”: the picnic basket further may contain one or more other items beside cheese and crackers.

The phrase “at least one” followed by a list of items joined by “and” denotes an item of the list but does not require every item of the list. Thus, “at least one of an apple and an orange” encompasses the following mutually exclusive scenarios: there is an apple but no orange; there is an orange but no apple; and there is both an apple and an orange. In these scenarios if there is an apple, there may be more than one apple, and if there is an orange, there may be more than one orange. Moreover, the phrase “one or more” followed by a list of items joined by “and” is the equivalent of “at least one” followed by the list of items joined by “and”.

Referring now to the drawings, one or more preferred embodiments of the invention are next described. The following description of one or more preferred embodiments is merely exemplary in nature and is in no way intended to limit the invention, its implementations, or uses.

Preferred embodiments in accordance with one or more aspects and features relate to apparatus comprising a packaged item, and systems and methods for packaging of the item. The four components forming part of the apparatus, systems and methods comprise (1) the item that is packaged; (2) a bag in which the textile item is contained; (3) an envelope in which the bag containing the textile item is enveloped; and (4) a tie that is used to secure the envelope. In the systems and methods, the textile item is received and contained within the bag; the bag is enveloped within the envelope; and the tie is used to secure the envelope with the bag completely enveloped therein.

The item preferably comprises a textile article used in foodservice such as an apron or head covering; or an article used in healthcare such as sanitary clothing worn by physicians, nurses, dentists and other workers involved in patient care (such as, for example, operating gowns and scrubs), or the sanitary clothing worn by patients (such as, for example, patient gowns and head coverings). The packaged item is designed to be highly effective in the protection and preservation of an aseptic environment for the textile items during transport, storage, and use.

The bag comprises a textile material. The bag defines an interior space configured to receive and completely contain the textile item within the interior space. The bag preferably has a single, open end defining a mouth through which the textile item is inserted into the bag. The mouth is closable for completely containing the item after being inserted through the mouth. The bag furthermore is configured to be closed to as to completely enclose the textile item within the interior space after insertion.

Textile or non-textile fasteners may be used to secure the bag in the closed state. The fasteners may comprise, for example, straps defined by textile materials that are joined or connected to the textile material of the bag defining the interior space. The straps may be connected or joined to the textile material of the bag by application of a chemical sealant; by ultrasonic welding; or by compression. Non-textile fasteners also may be used such as buckles which are attached to the material of the bag defining the interior space, and such attachment by include use of textile materials. Such non-textile fasteners may be made by stamping, printing, or injection molding, or combinations thereof. Nonetheless, in all of these embodiments, the components should remain reusable in accordance with the present invention regardless of the fasteners.

The envelope also comprises a sheet of textile material. The sheet is dimensioned such that the bag with the textile item contained therein can be entirely enveloped by folding the sheet about the bag containing the textile item.

The tie comprises a piece of textile material preferably in the form of a strap. The strap is of sufficient length to wrap completely around the sheet in order to secure the sheet with the bag enveloped therein. In at least some embodiments, the tie includes non-textile fasteners for securing the tie in this wrapped state. The non-textile fasteners may comprise buckles and may be made by stamping, printing, or injection molding, or combinations thereof. Nonetheless, in all of the embodiments, the components should remain reusable in accordance with the present invention regardless of the fasteners.

Within this context, a “reusable component” means one of the aforementioned four components each of which comprises a textile material and that, after its intended use, the component is able to be sterilized and used again in the same way in accordance with its “initial purpose”, the component having been contaminated and then returned to an uncontaminated state through sterilization. Methods of sterilization in accordance with one or more aspects and features of the invention preferably comprise washing, heating, and combinations of washing and heating of the components. A reusable component preferably is reusable many times and, furthermore, preferably is recyclable or capable of being repurposed for a different use other than its initial purpose at the end of its initial usable life.

Additionally, a “bag” is synonymous with “pouch” and, as used herein, means a flexible container having an opening that is closable for containing a textile item therein. The container may have a single opening defining a mouth that is closable or two openings defining top and bottom mouths that are closable. Furthermore, the container is configured for opening of a closed mouth and inverting the container for removing or “shaking out” of the textile item contained therein without actually touching the textile item.

In packaging the textile item, the sheet is used that is configured to be folded so as to encompass and completely envelope the textile item, and the strap is used to secure the folded sheet with the textile item encompassed within the folded sheet. Both the sheet and strap are reusable as is the textile item that is packaged. Furthermore, the sheet and strap preferably are made from the same material, and the textile item may be made from the same textile material.

Preferred material comprise nylon, polyester, cotton, flax, hemp. The textile materials furthermore preferably are hydrophobic so as to be repellant, semi-impermeable, or impermeable to fluid such as water or alcohol. Preferably, a chemical coating is applied to the materials of which the textiles are made such as, for example, a member of the C6 fluorocarbon family or C6 fluorotelomers.

In one or more embodiments, a polyester material is coated with a water and alcohol proof agent from the C6 fluorocarbon family.

In one or more embodiments, Texfin ND-C6 is applied to a textile material, which coating is known to provide a high-quality finishing of cellulosic and synthetic performance textiles to impart durable oil, water and stain repellency.

In one or more embodiments, the textile material of the textile item, the sheet defining the envelope, the bag, and combinations thereof is 99% or more polyester woven fabric with less than 1% carbon fiber treated with a C6 fluorocarbon to confer additional fluid resistance, which has been certified as a compliant material for reusable packaging for one hundred sterilization cycles, including washing and drying.

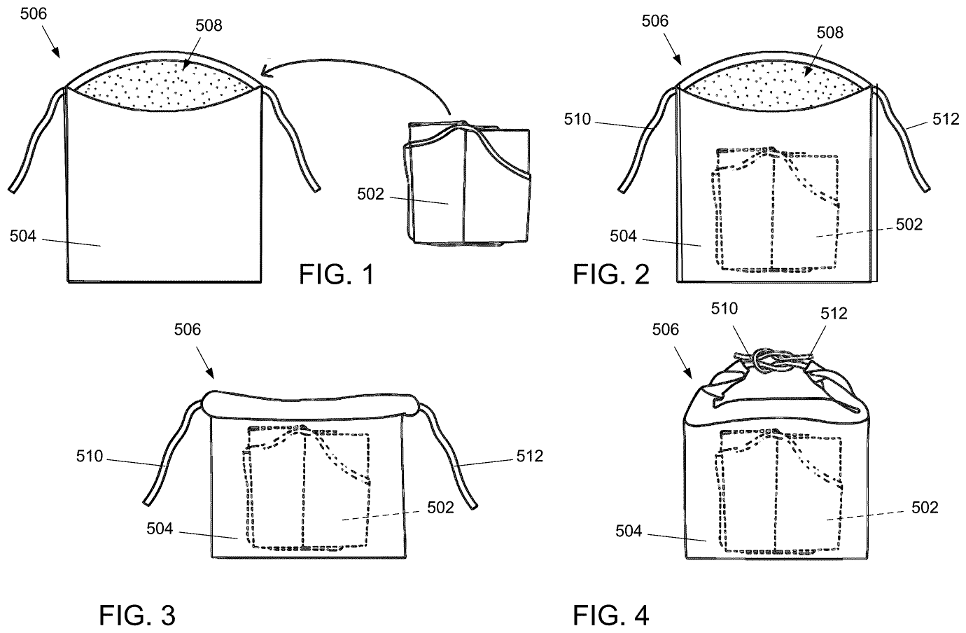

Turning now to the drawings, FIGS. 1-4 illustrate the insertion of a sterilized textile item 502 into an interior space of a bag 504 and closing an open end 506 of the bag to retain the textile item therein. In accordance with aspects and features of the invention, the bag comprises a sterilized textile material defining the interior space of the bag, and the bag is flexible and comprises a textile material that preferably is the same as the textile material of the textile item. The resulting package preferably constitutes mono material packaging.

Specifically, FIG. 1 illustrates the insertion of the sterilized textile item through the open end 506 of the bag defining a mouth 508 of the bag into the interior space of the bag 504.

FIG. 2 shows the textile item 502 completely contained within the interior space of the bag.

FIG. 3 shows the closing of the mouth of the end 506 of the bag by folding or rolling the end of the bag, and FIG. 4 shows the securement of the bag in the closed configuration by tying together opposite sides of the rolled or folded end of the bag using straps 510,512 that are attached to the opposite sides of the bag.

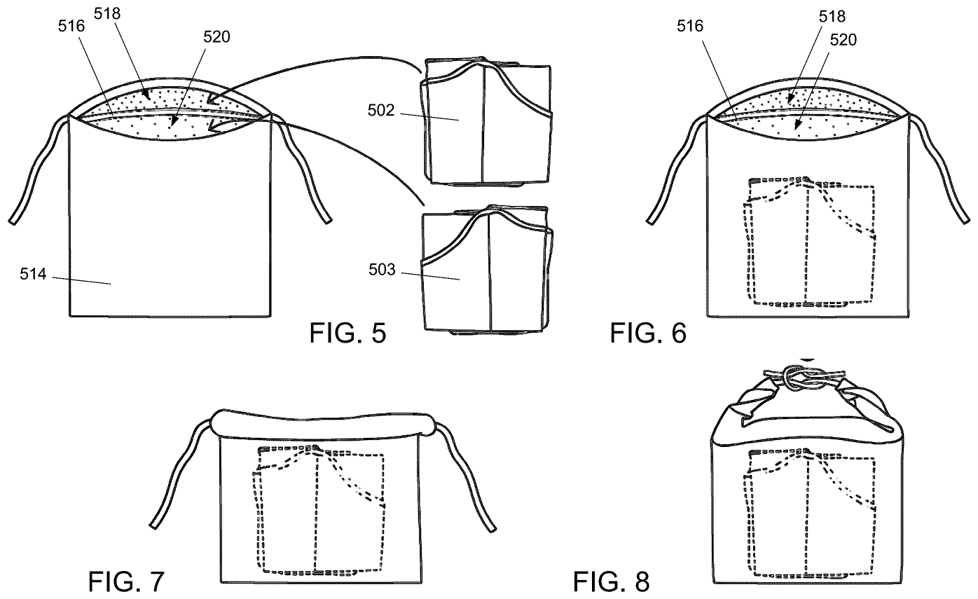

FIGS. 5-8 are similar to those of FIGS. 1-4 and differ in that these figures illustrate the insertion of multiple sterilized textile items 502,503 into an interior space of a bag 514 that has a partitioning wall 516 that divides the interior space into two compartments 518,520. In accordance with aspects and features of the invention, the bag comprises a sterilized textile material defining the interior space of the bag, and the bag is flexible and comprises a textile material that preferably is the same as the textile material of the textile items. The resulting package preferably constitutes mono material packaging.

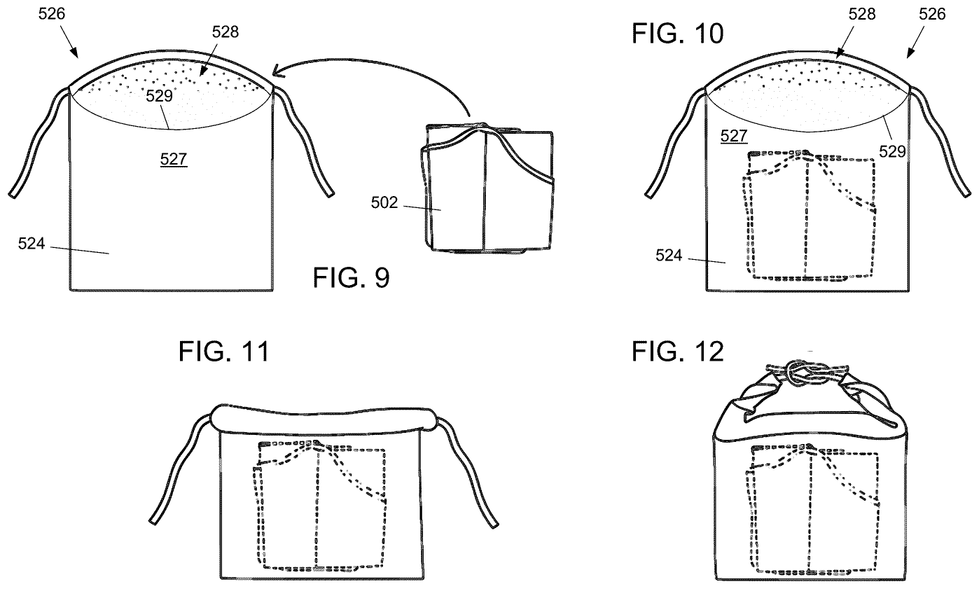

FIGS. 9-12 are similar to those of FIGS. 1-4 and differ in that a bag 524 comprises an open end 526 having a front wall 527 that does not extend as far upwardly so as to define a scalloped top edge 529. The resulting scalloped mouth 528 is believed to facilitate insertion of the textile item 502 through the mouth into the interior space of the bag.

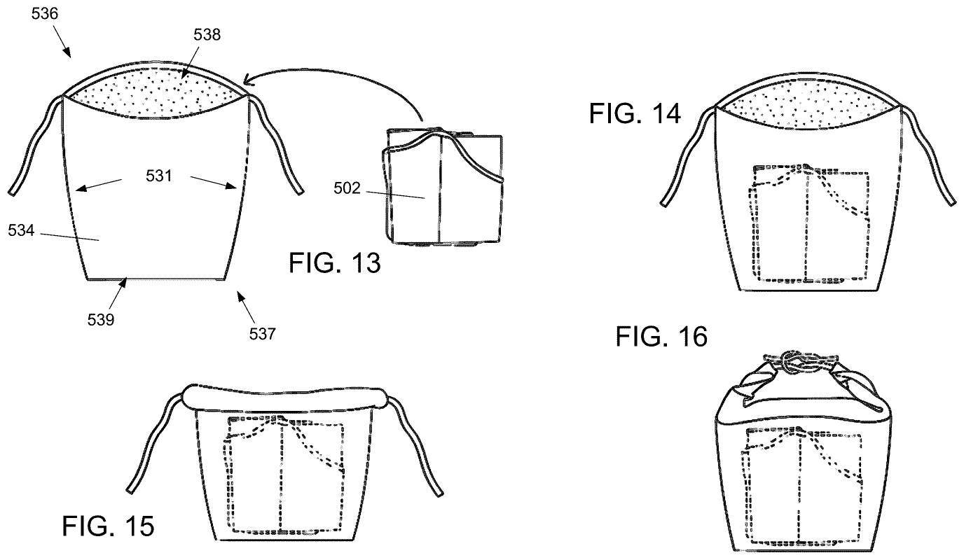

FIGS. 13-16 are similar to those of FIGS. 1-4 and differ in that a bag 534 is wider at the open end 536 defining the mouth 538 than at an opposite end 537 defining a bottom 539 of the bag 534. The surface of the bag 534 is curved in its extent 531 from the first end 536 to the opposite end 537. This shape of the bag is believed to create a funnel effect that enhances entry into and exit from the bag of the textile item 502.

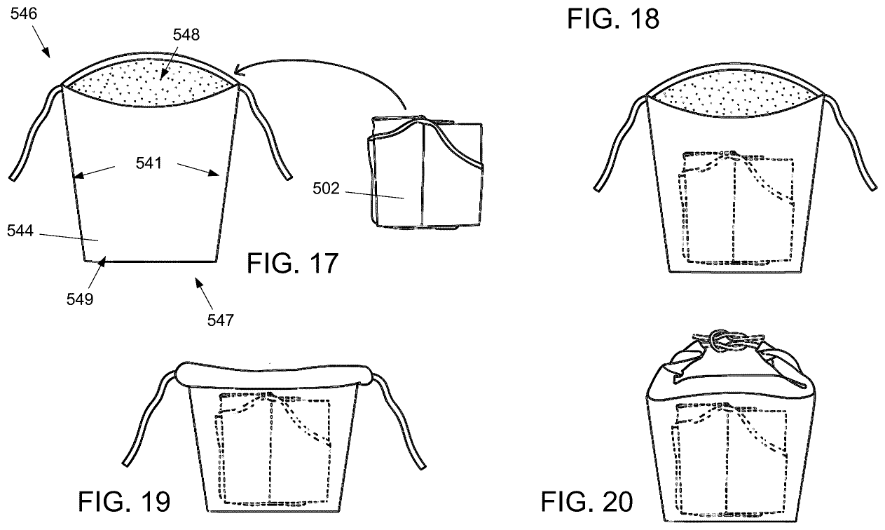

FIGS. 17-20 are similar to those of FIGS. 1-4 and differ in that a bag 544 is wider at the open end 546 defining the mouth 548 than at an opposite end 547 defining a bottom 549 of the bag 544. The surface of the bag 544 is curved in its extent 541 from the first end 546 to the opposite end 547. This shape of the bag is believed to create a funnel effect that enhances entry into and exit from the bag of the textile item 502.

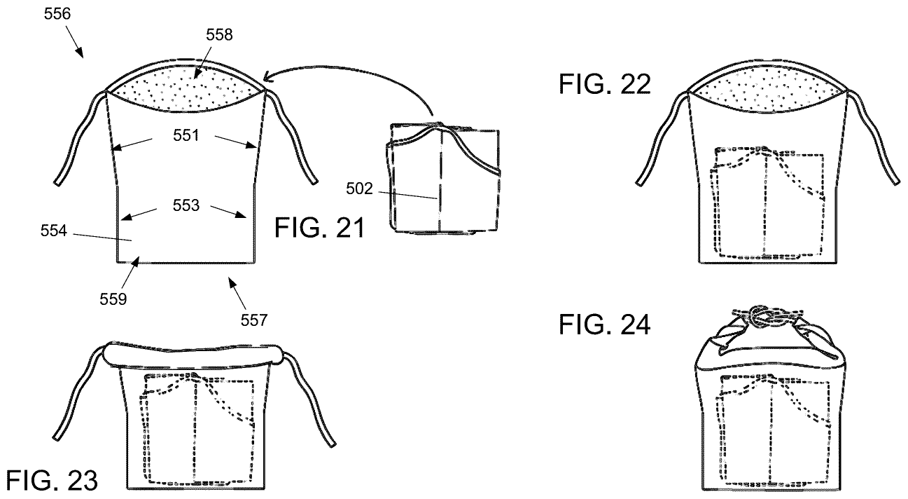

FIGS. 21-24 are similar to those of FIGS. 1-4 and differ in that a bag 554 is wider at the open end 556 defining the mouth 558 than at an opposite end 557 defining a bottom 559 of the bag 554. The surface of the bag 554 is linear in a first extent 551 from the first end 556 toward the opposite end 557, wherein its width decreases, and is linear in a second, remaining extent 553 to the opposite end 557, wherein the width is fixed. This shape of the bag is believed to create a funnel effect that enhances entry into and exit from the bag of the textile item 502.

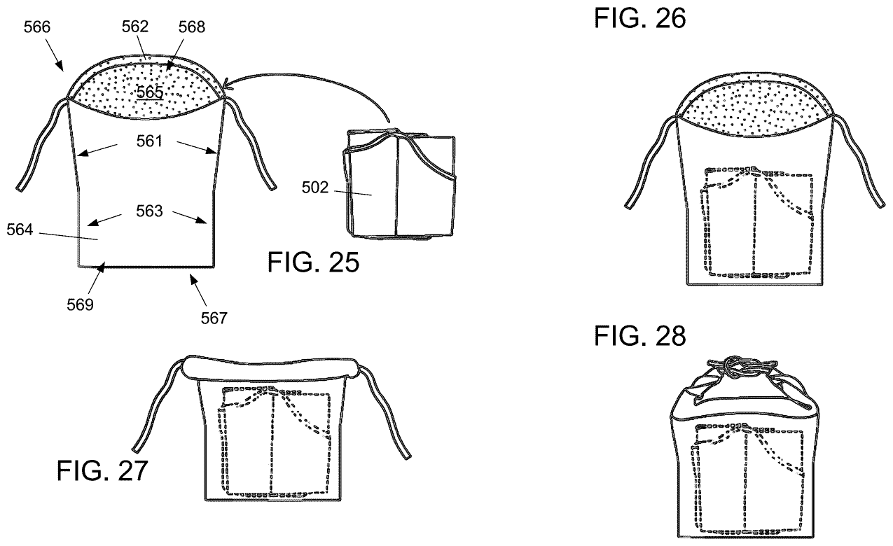

FIGS. 25-28 are similar to those of FIGS. 1-4 and differ in that a bag 564 is wider at the open end 566 defining the mouth 568 than at an opposite end 567 defining a bottom 569 of the bag 564. The surface of the bag 564 is linear in a first extent 561 from the first end 566 toward the opposite end 567, wherein its width decreases, and is linear in a second, remaining extent 563 to the opposite end 567, wherein the width is fixed. Moreover, open end 566 has a back wall 565 that extend further upwardly so as to define a scalloped top edge 562. The resulting scalloped mouth 568 is believed to facilitate insertion of the textile item 502 through the mouth into the interior space of the bag, and the shape of the bag is believed to create a funnel effect that enhances entry into and exit from the bag of the textile item 502.

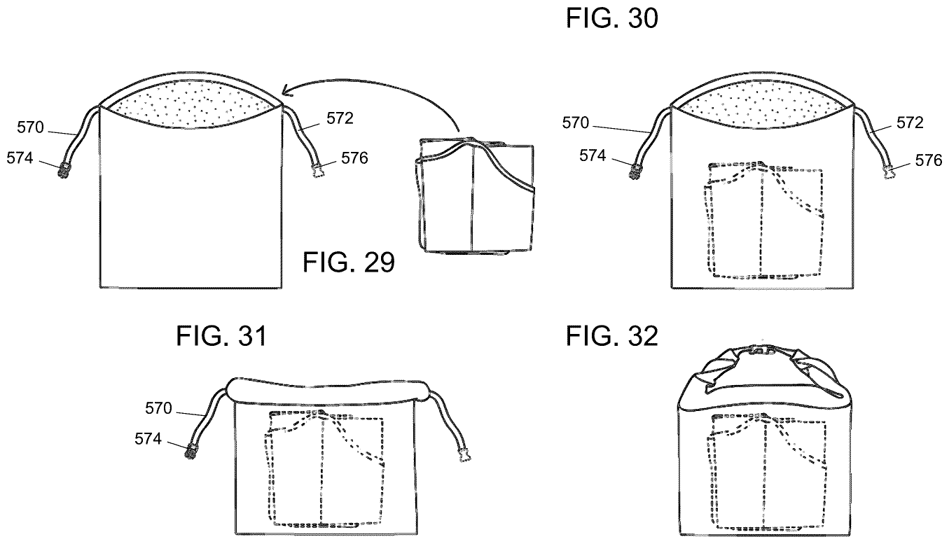

FIGS. 29-32 are similar to those of FIGS. 1-4 and differ in that straps 570,572 have fasteners in the form of buckles 584,586 by which the bag is secured in the closed configuration.

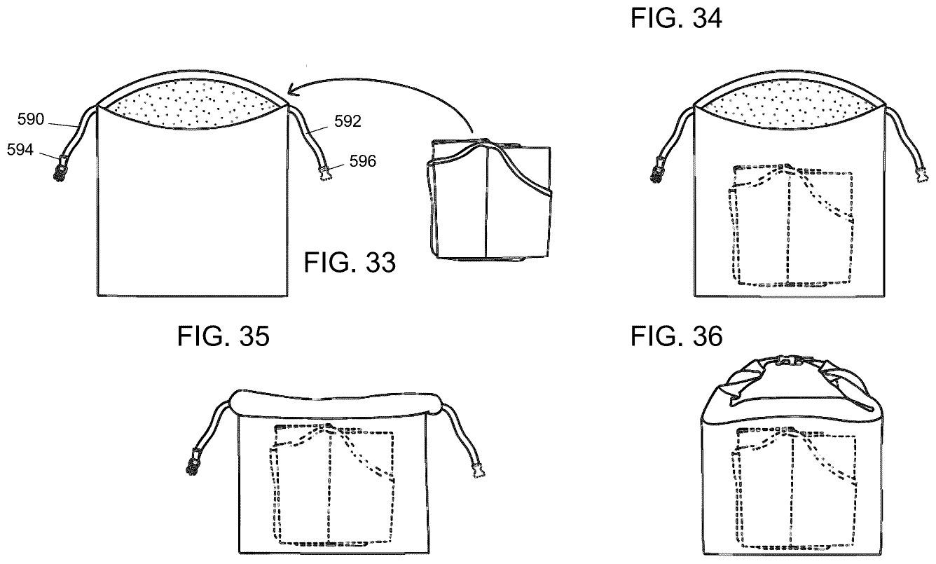

FIGS. 33-36 are similar to those of FIGS. 1-4 and differ in that straps 590,592 have fasteners in the form of buckles 594,596 by which the bag is secured in the closed configuration, wherein the strap length between the buckle 594 and the attachment of the strap 590 to a side of the bag is adjustable.

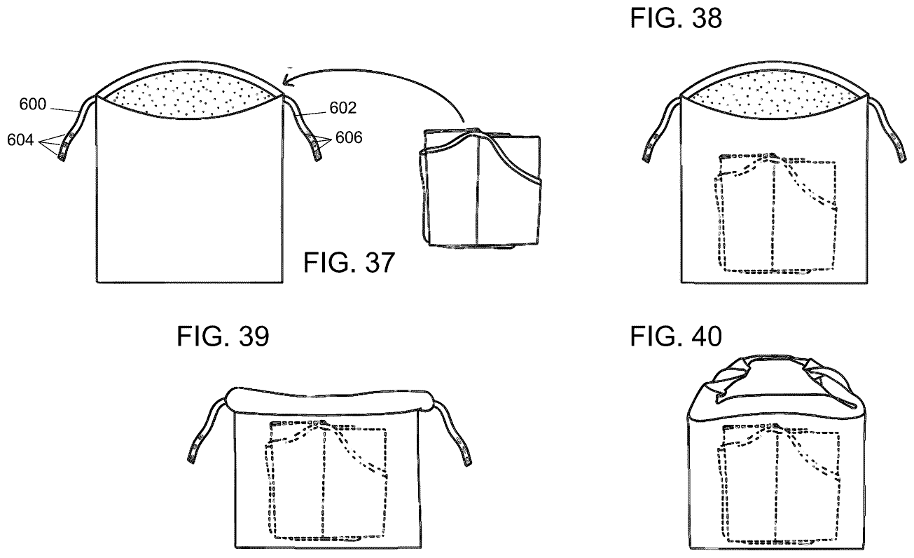

FIGS. 37-40 are similar to those of FIGS. 1-4 and differ in that straps 600,602 have fasteners in the form of buttons 604 and buttonholes 606 by which the bag is secured in the closed configuration.

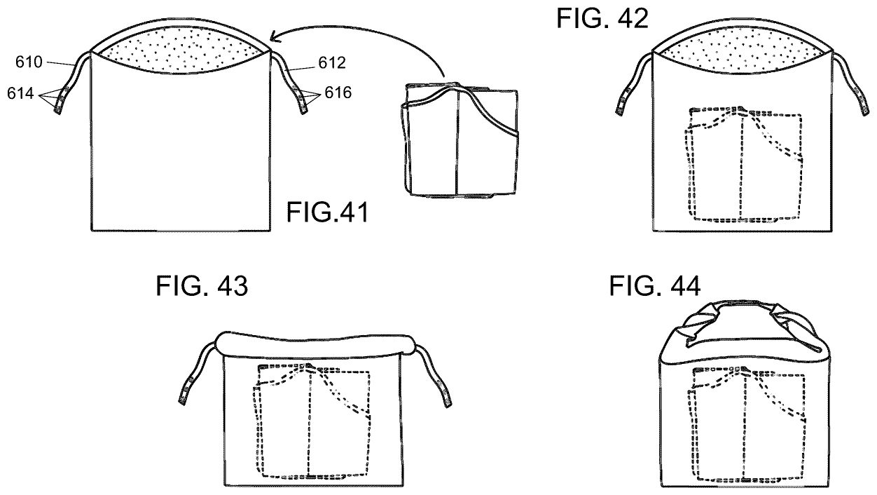

FIGS. 41-44 are similar to those of FIGS. 1-4 and differ in that straps 610,612 have fasteners in the form of interlocking discs 614,606 by which the bag is secured in the closed configuration. The interlocking discs are sometimes called snap fastener, snap buttons, press studs, press fasteners, dome fasteners, poppers, and are a pair of interlocking discs made out of a metal or plastic, that are commonly used in place of traditional buttons to fasten clothing and for similar purposes.

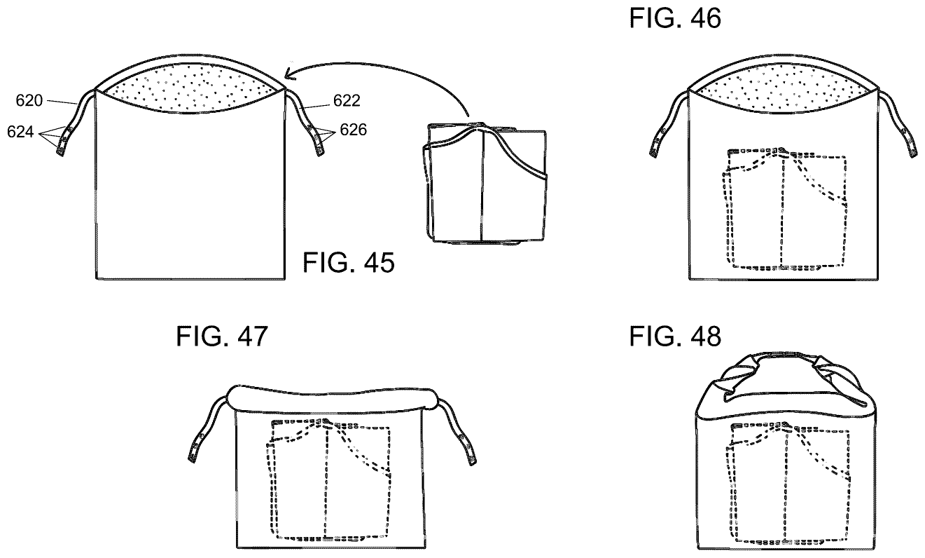

FIGS. 45-48 are similar to those of FIGS. 1-4 and differ in that straps 620,622 have fasteners in the form of studs 624 with corresponding backs 626 by which the bag is secured in the closed configuration. The studs and backs are like earring fasteners.

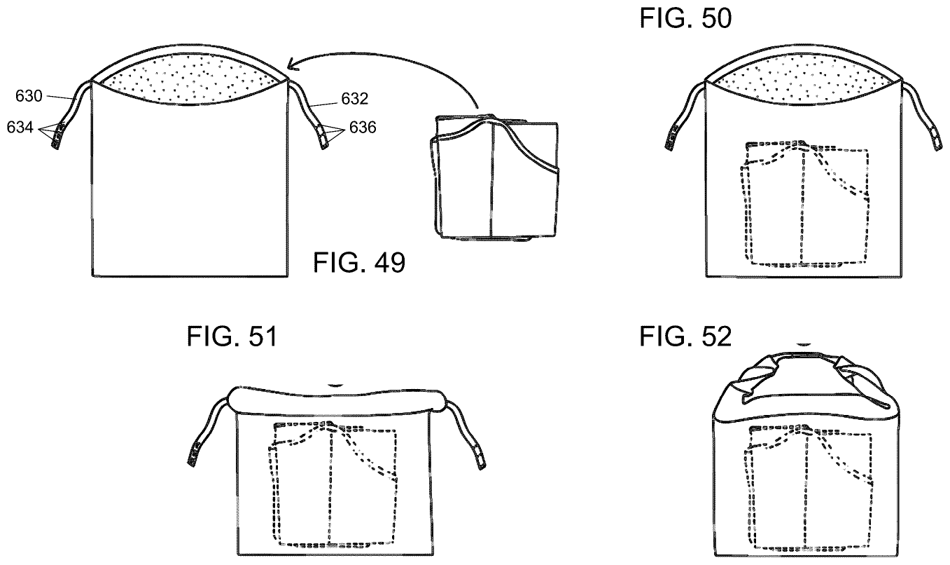

FIGS. 49-52 are similar to those of FIGS. 1-4 and differ in that straps 630,632 have fasteners in the form of hooks 634 and eyes 636 by which the bag is secured in the closed configuration.

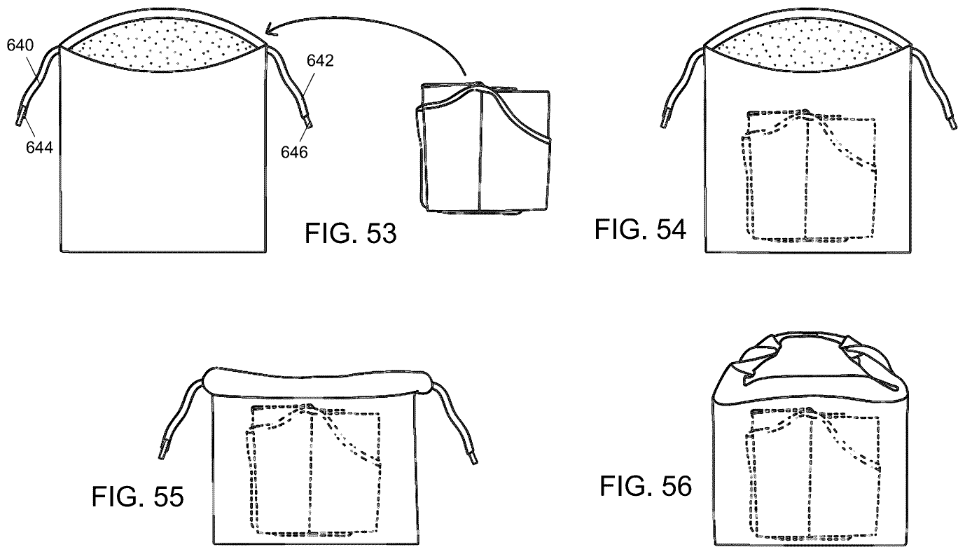

FIGS. 53-56 are similar to those of FIGS. 1-4 and differ in that straps 640,642 have fasteners in the form of single or double-sided dissolvable tape 644,646—or other dissolvable adhesive—by which the bag is secured in the closed configuration.

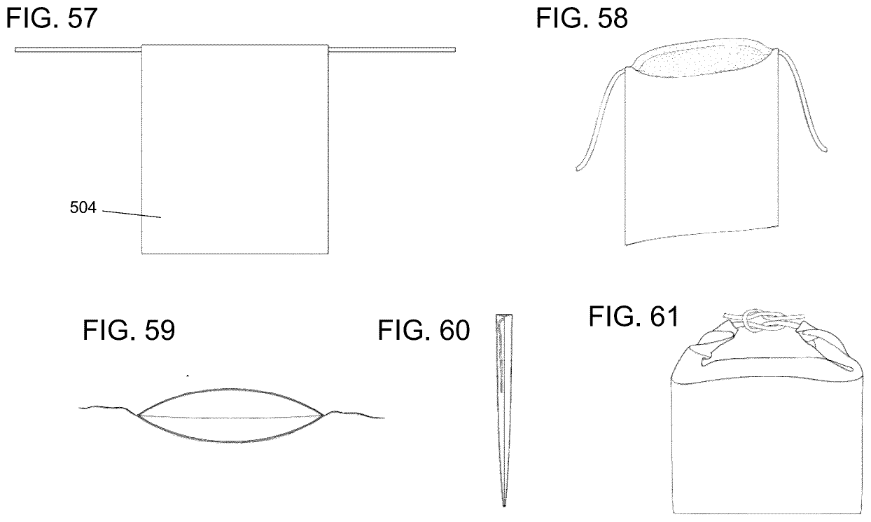

FIGS. 57-61 are additional views of the bag 504. Specifically, FIG. 57 is a front elevational view of the bag 504. FIG. 58 is a top perspective view of the bag 504. FIG. 59 is a top plan view of the bag 504. FIG. 60 is a side elevational view of the bag 504, and FIG. 61 is a front elevational view of the bag 504 showing the securement of the bag in the closed configuration.

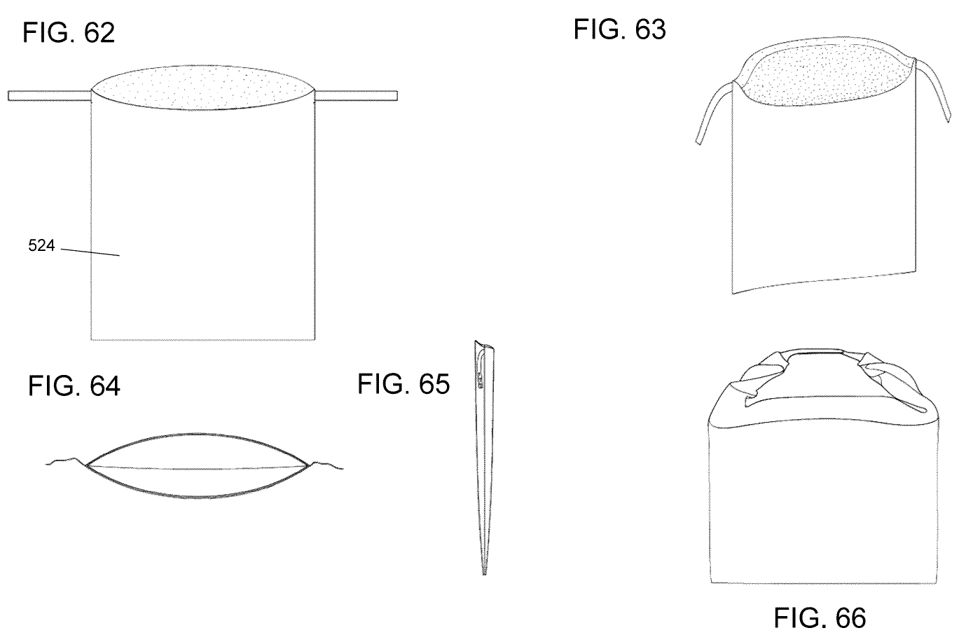

FIGS. 62-66 are additional views of the bag 524. Specifically, FIG. 62 is a front elevational view of the bag 524. FIG. 63 is a top perspective view of the bag 524. FIG. 64 is a top plan view of the bag 524. FIG. 65 is a side elevational view of the bag 524, and FIG. 66 is a front elevational view of the bag 524 showing the securement of the bag in the closed configuration.

FIGS. 67-71 are additional views of the bag 534. Specifically, FIG. 67 is a front elevational view of the bag 534. FIG. 68 is a top perspective view of the bag 534. FIG. 69 is a top plan view of the bag 534. FIG. 70 is a side elevational view of the bag 534, and FIG. 71 is a front elevational view of the bag 534 showing the securement of the bag in the closed configuration.

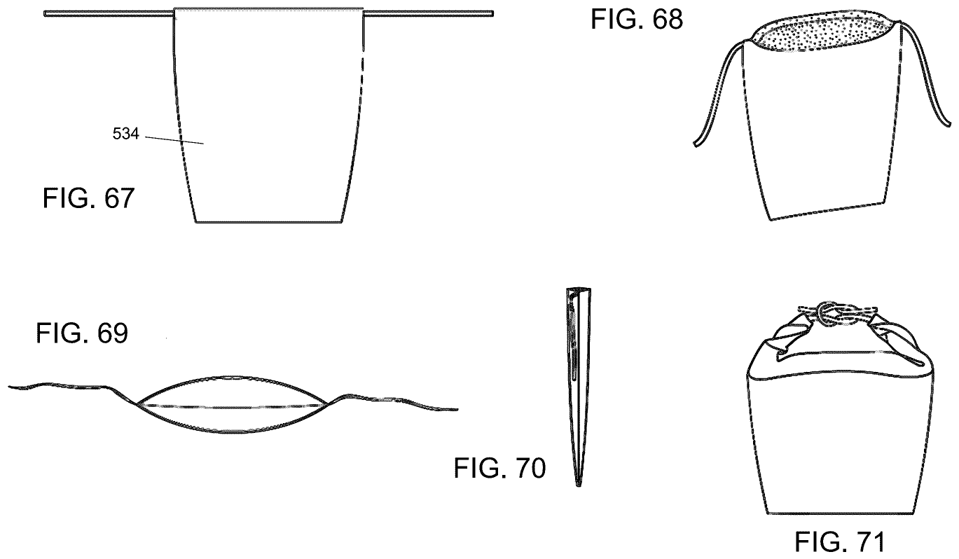

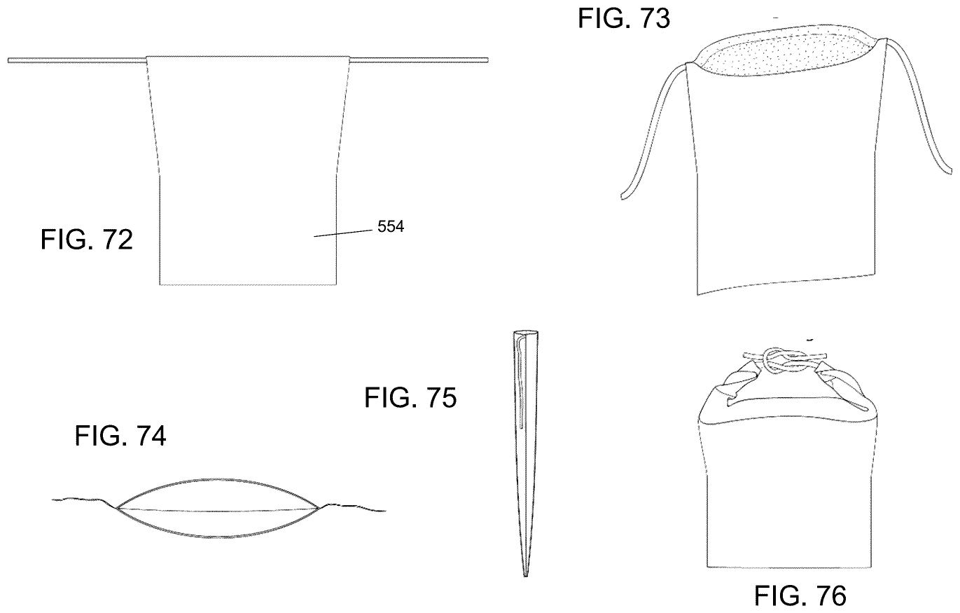

FIGS. 72-76 are additional views of the bag 554. Specifically, FIG. 72 is a front elevational view of the bag 554. FIG. 73 is a top perspective view of the bag 554. FIG. 74 is a top plan view of the bag 554. FIG. 75 is a side elevational view of the bag 554, and FIG. 76 is a front elevational view of the bag 554 showing the securement of the bag in the closed configuration.

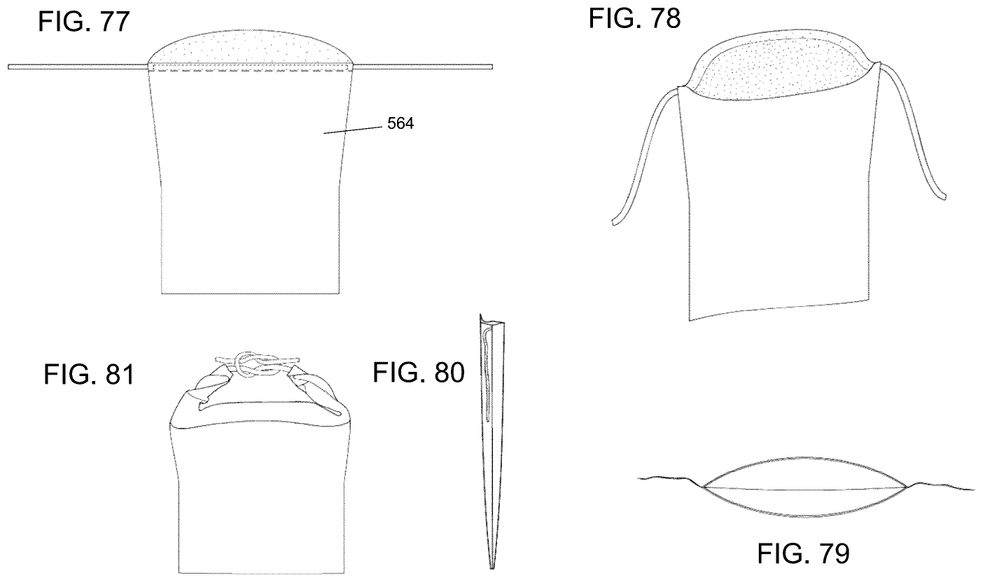

FIGS. 77-79 are additional views of the bag 564. Specifically, FIG. 77 is a front elevational view of the bag 564. FIG. 78 is a top perspective view of the bag 564. FIG. 79 is a top plan view of the bag 564. FIG. 80 is a side elevational view of the bag 564, and FIG. 81 is a front elevational view of the bag 564 showing the securement of the bag in the closed configuration.

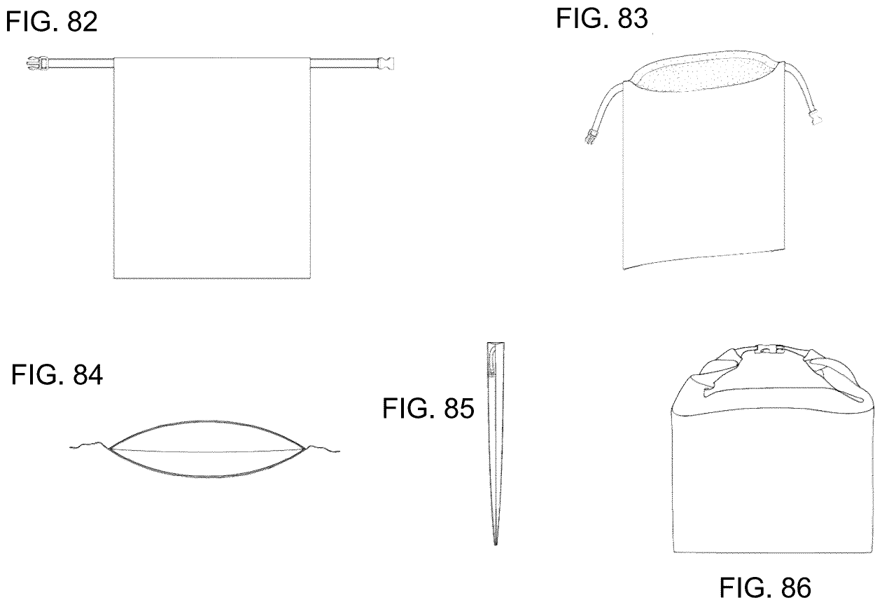

FIGS. 82-86 are additional views of the bag of FIG. 29. Specifically, FIG. 82 is a front elevational view of the bag of FIG. 29. FIG. 83 is a top perspective view of the bag of FIG. 29. FIG. 84 is a top plan view of the bag of FIG. 29. FIG. 85 is a side elevational view of the bag of FIG. 29, and FIG. 86 is a front elevational view of the bag of FIG. 29 showing the securement of the bag in the closed configuration.

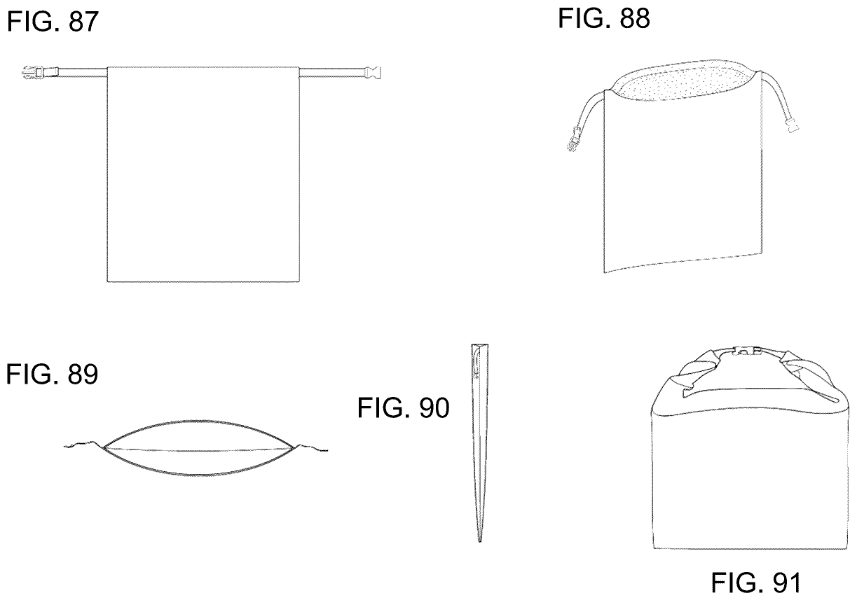

FIGS. 87-91 are additional views of the bag of FIG. 33. Specifically, FIG. 87 is a front elevational view of the bag of FIG. 33. FIG. 88 is a top perspective view of the bag of FIG. 33. FIG. 89 is a top plan view of the bag of FIG. 33. FIG. 90 is a side elevational view of the bag of FIG. 33, and FIG. 91 is a front elevational view of the bag of FIG. 33 showing the securement of the bag in the closed configuration.

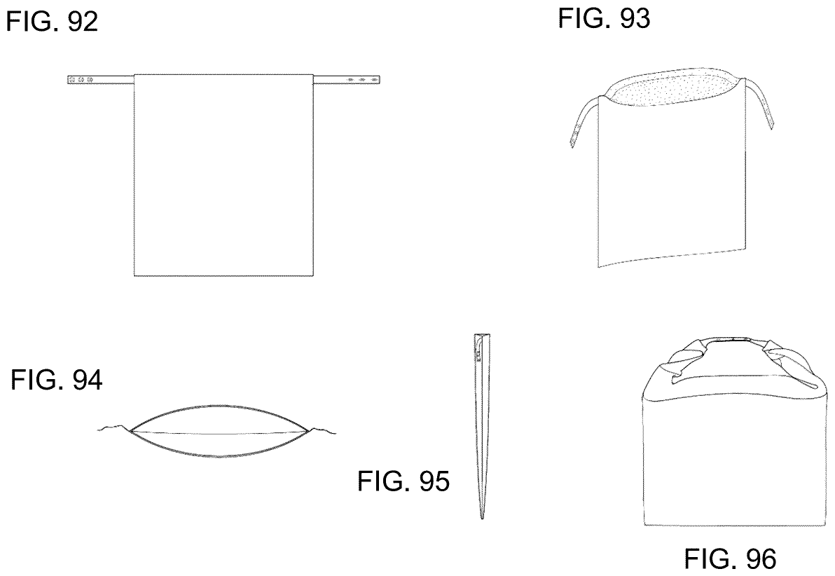

FIGS. 92-96 are additional views of the bag of FIG. 37. Specifically, FIG. 92 is a front elevational view of the bag of FIG. 37. FIG. 93 is a top perspective view of the bag of FIG. 37. FIG. 94 is a top plan view of the bag of FIG. 37. FIG. 95 is a side elevational view of the bag of FIG. 37, and FIG. 96 is a front elevational view of the bag of FIG. 37 showing the securement of the bag in the closed configuration.

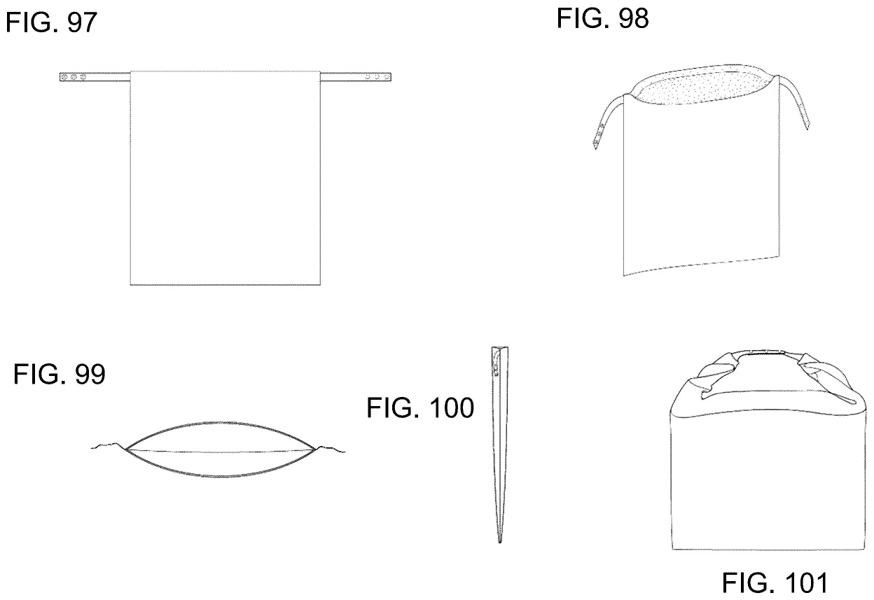

FIGS. 97-101 are additional views of the bag of FIG. 41. Specifically, FIG. 97 is a front elevational view of the bag of FIG. 41. FIG. 98 is a top perspective view of the bag of FIG. 41. FIG. 99 is a top plan view of the bag of FIG. 41. FIG. 100 is a side elevational view of the bag of FIG. 41, and FIG. 101 is a front elevational view of the bag of FIG. 41 showing the securement of the bag in the closed configuration.

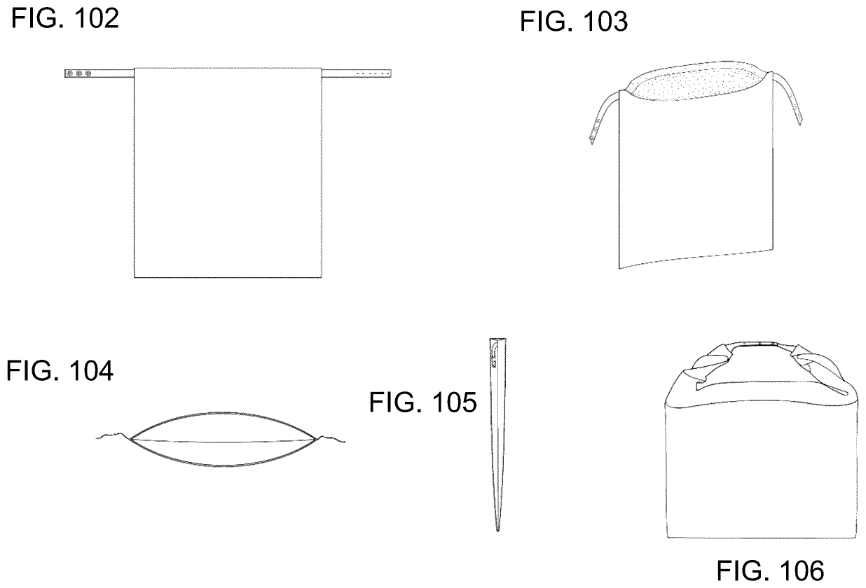

FIGS. 102-106 are additional views of the bag of FIG. 45. Specifically, FIG. 102 is a front elevational view of the bag of FIG. 45. FIG. 103 is a top perspective view of the bag of FIG. 45. FIG. 104 is a top plan view of the bag of FIG. 45. FIG. 105 is a side elevational view of the bag of FIG. 45, and FIG. 106 is a front elevational view of the bag of FIG. 45 showing the securement of the bag in the closed configuration.

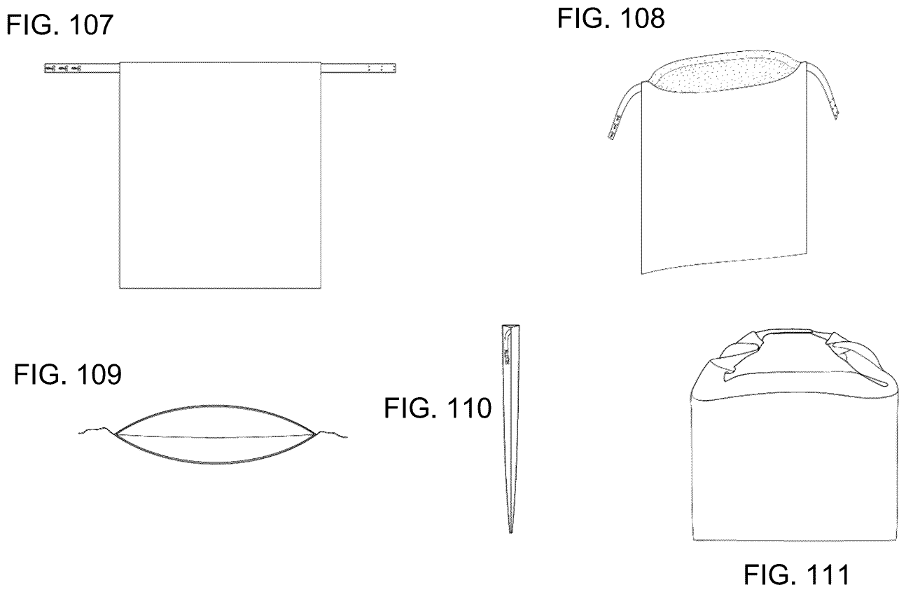

FIGS. 107-111 are additional views of the bag of FIG. 49. Specifically, FIG. 107 is a front elevational view of the bag of FIG. 49. FIG. 108 is a top perspective view of the bag of FIG. 49. FIG. 109 is a top plan view of the bag of FIG. 49. FIG. 110 is a side elevational view of the bag of FIG. 49, and FIG. 111 is a front elevational view of the bag of FIG. 49 showing the securement of the bag in the closed configuration.

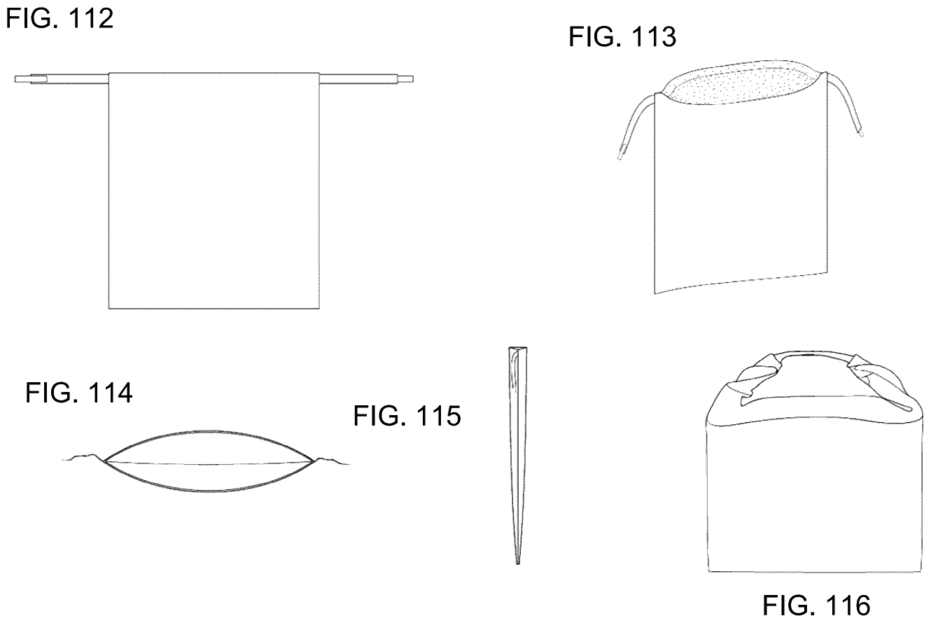

FIGS. 112-116 are additional views of the bag of FIG. 53. Specifically, FIG. 112 is a front elevational view of the bag of FIG. 53. FIG. 113 is a top perspective view of the bag of FIG. 53. FIG. 114 is a top plan view of the bag of FIG. 53. FIG. 115 is a side elevational view of the bag of FIG. 53, and FIG. 116 is a front elevational view of the bag of FIG. 53 showing the securement of the bag in the closed configuration.

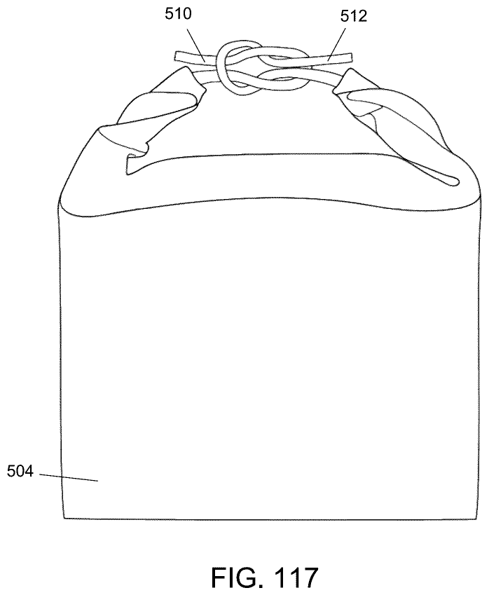

FIG. 117 is an enlarged view of the bag of FIG. 4 showing the tying of the straps in a bow or a reef knot.

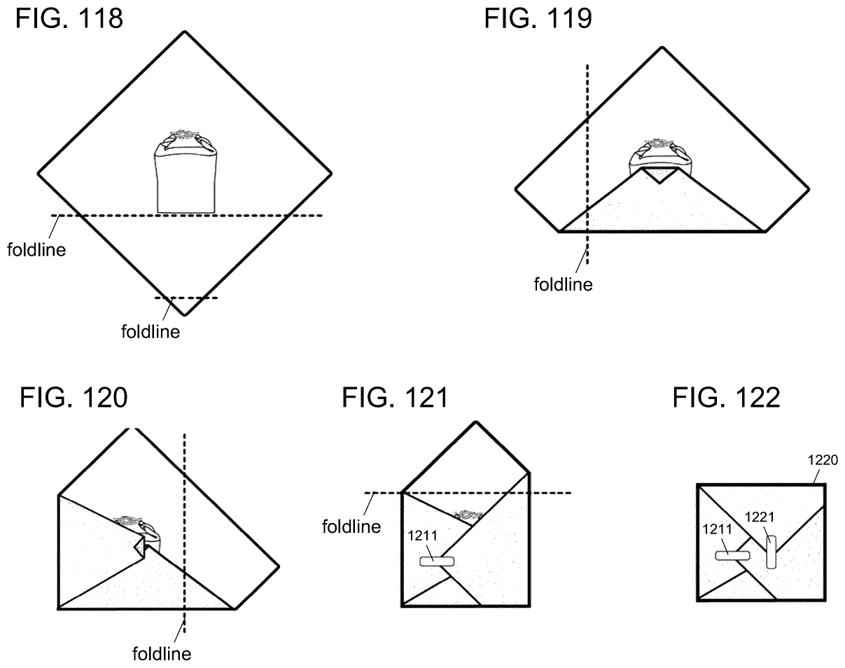

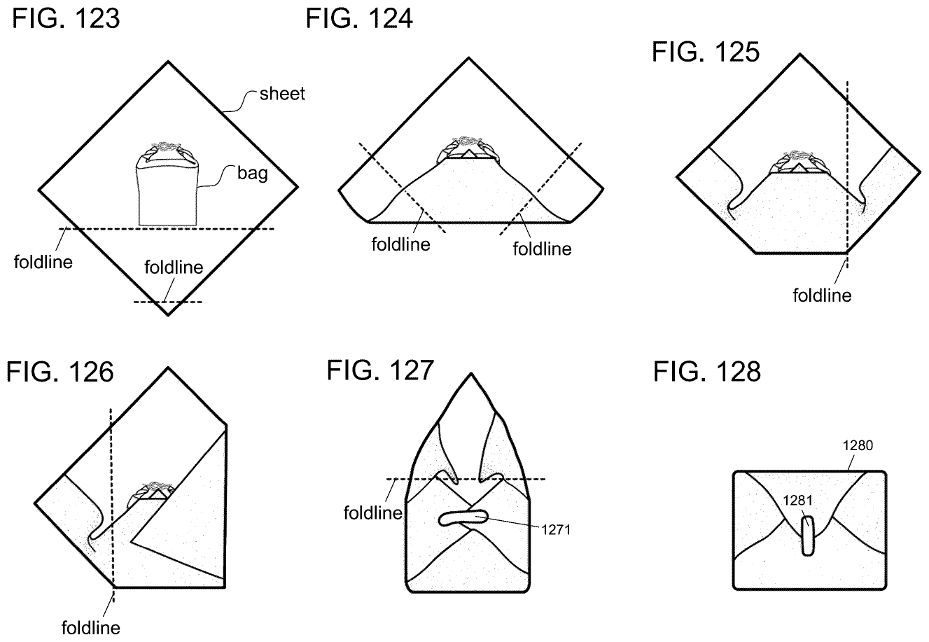

FIGS. 118-122 illustrate steps of a preferred method of enveloping a bag secured in a closed configuration by folding a sheet around the bag to entirely envelope the bag, in accordance with one or more aspects and features of the invention. Dissolvable tape 1211,1221 or other dissolvable adhesive may be used as seen in FIGS. 121 and 122 for at least temporarily holding the shape of the resulting envelope 1220.

FIGS. 123-128 illustrate steps of another preferred method of enveloping a bag secured in a closed configuration by folding a sheet around the bag to entirely envelope the bag, in accordance with one or more aspects and features of the invention. Dissolvable tape 1271,1281 or other dissolvable adhesive may be used as seen in FIGS. 127 and 128 for at least temporarily holding the shape of the resulting envelope 1280.

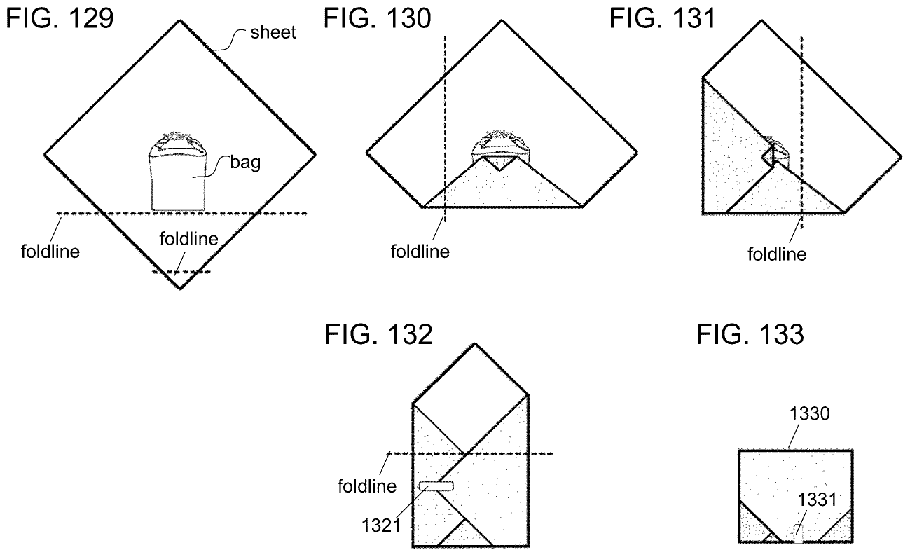

FIGS. 129-133 illustrate steps of a method of enveloping a bag secured in a closed configuration by folding a sheet around the bag to entirely envelope the bag, in accordance with one or more aspects and features of the invention. Dissolvable tape 1321,1331 or other dissolvable adhesive may be used as seen in FIGS. 132 and 133 for at least temporarily holding the shape of the resulting envelope 1330.

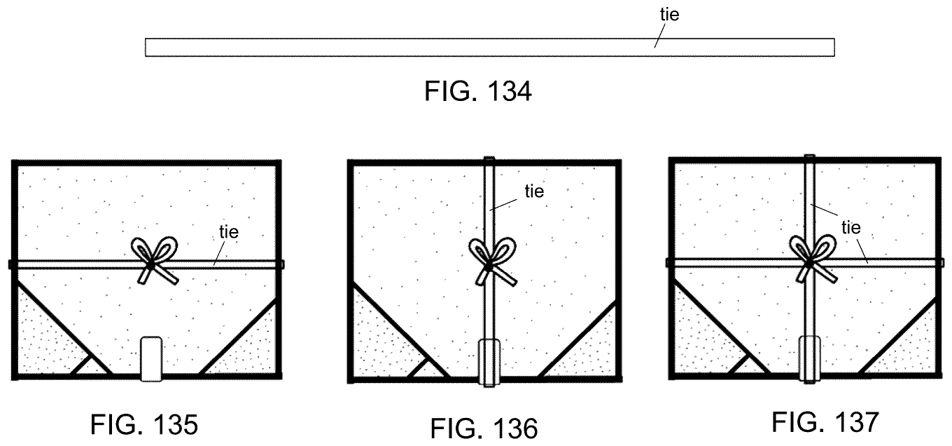

FIG. 134 is a top plan view of a preferred tie that may be used to secure the envelope of FIG. 133, in accordance with one or more aspects and features of the invention. Here, the ends of the tie are tied together in a form of a knot.

FIG. 135 is a top plan view of the tie of FIG. 134 used in a first preferred manner to secure the envelope of FIG. 133, in accordance with one or more aspects and features of the invention.

FIG. 136 is a top plan view of the tie of FIG. 134 used in a second preferred manner to secure the envelope of FIG. 133, in accordance with one or more aspects and features of the invention.

FIG. 137 is a top plan view of the tie of FIG. 134 used in a third preferred manner to secure the envelope of FIG. 133, in accordance with one or more aspects and features of the invention.

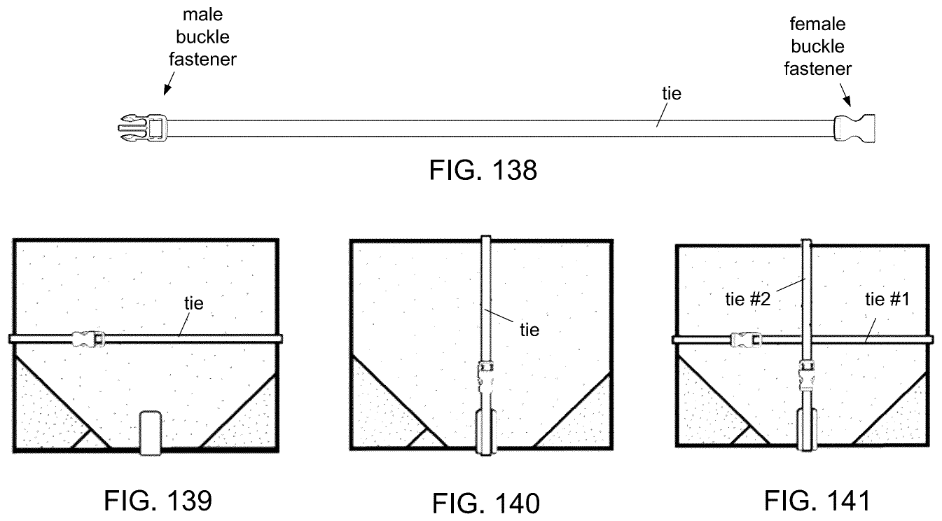

FIG. 138 is a top plan view of another preferred tie that may be used to secure the envelope of FIG. 133, in accordance with one or more aspects and features of the invention.

FIG. 139 is a top plan view of the tie of FIG. 138 used in a first preferred manner to secure the envelope of FIG. 133, in accordance with one or more aspects and features of the invention.

FIG. 140 is a top plan view of the tie of FIG. 138 used in a second preferred manner to secure the envelope of FIG. 133, in accordance with one or more aspects and features of the invention.

FIG. 141 is a top plan view of two ties of FIG. 138 used in a preferred manner to secure the envelope of FIG. 133, in accordance with one or more aspects and features of the invention.

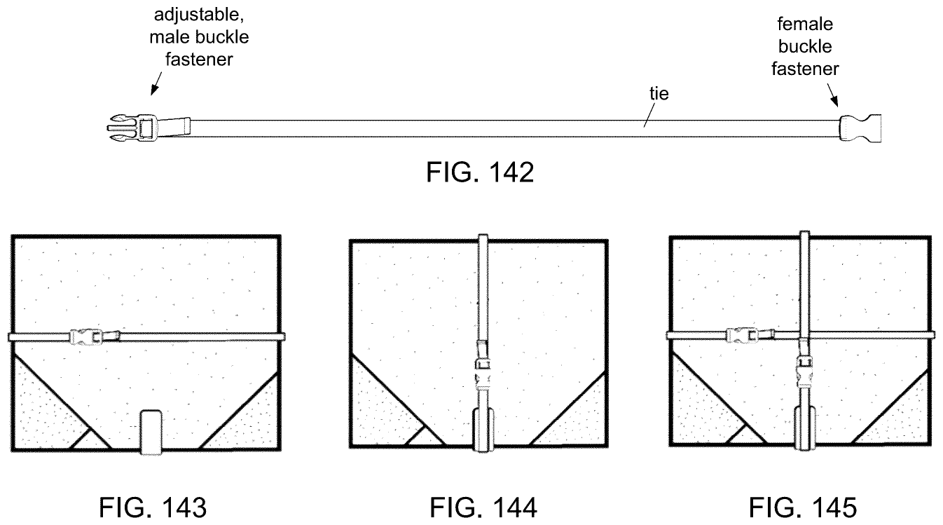

FIG. 142 is a top plan view of another preferred tie that may be used to secure the envelope of FIG. 133, in accordance with one or more aspects and features of the invention.

FIG. 143 is a top plan view of the tie of FIG. 142 used in a first preferred manner to secure the envelope of FIG. 133, in accordance with one or more aspects and features of the invention.

FIG. 144 is a top plan view of the tie of FIG. 142 used in a second preferred manner to secure the envelope of FIG. 133, in accordance with one or more aspects and features of the invention.

FIG. 145 is a top plan view of two ties of FIG. 142 used in a preferred manner to secure the envelope of FIG. 133, in accordance with one or more aspects and features of the invention.

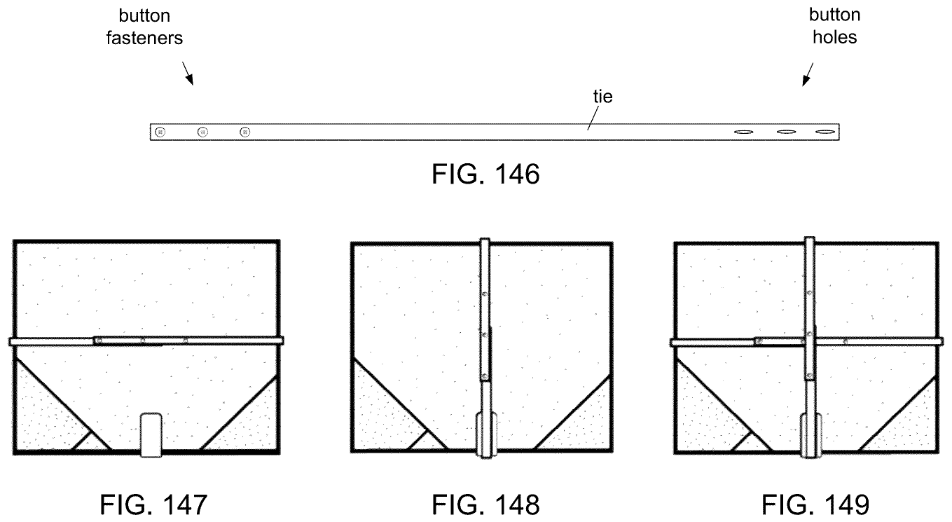

FIG. 146 is a top plan view of another preferred tie that may be used to secure the envelope of FIG. 133, in accordance with one or more aspects and features of the invention.

FIG. 147 is a top plan view of the tie of FIG. 146 used in a first preferred manner to secure the envelope of FIG. 133, in accordance with one or more aspects and features of the invention.

FIG. 148 is a top plan view of the tie of FIG. 146 used in a second preferred manner to secure the envelope of FIG. 133, in accordance with one or more aspects and features of the invention.

FIG. 149 is a top plan view of two ties of FIG. 146 used in a preferred manner to secure the envelope of FIG. 133, in accordance with one or more aspects and features of the invention.

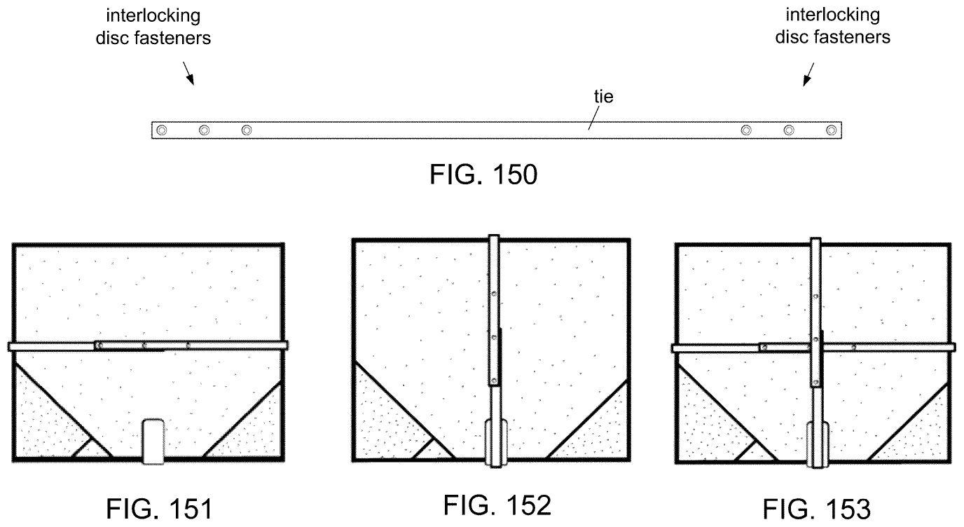

FIG. 150 is a top plan view of another preferred tie that may be used to secure the envelope of FIG. 133, in accordance with one or more aspects and features of the invention.

FIG. 151 is a top plan view of the tie of FIG. 150 used in a first preferred manner to secure the envelope of FIG. 133, in accordance with one or more aspects and features of the invention.

FIG. 152 is a top plan view of the tie of FIG. 150 used in a second preferred manner to secure the envelope of FIG. 133, in accordance with one or more aspects and features of the invention.

FIG. 153 is a top plan view of two ties of FIG. 150 used in a preferred manner to secure the envelope of FIG. 133, in accordance with one or more aspects and features of the invention.

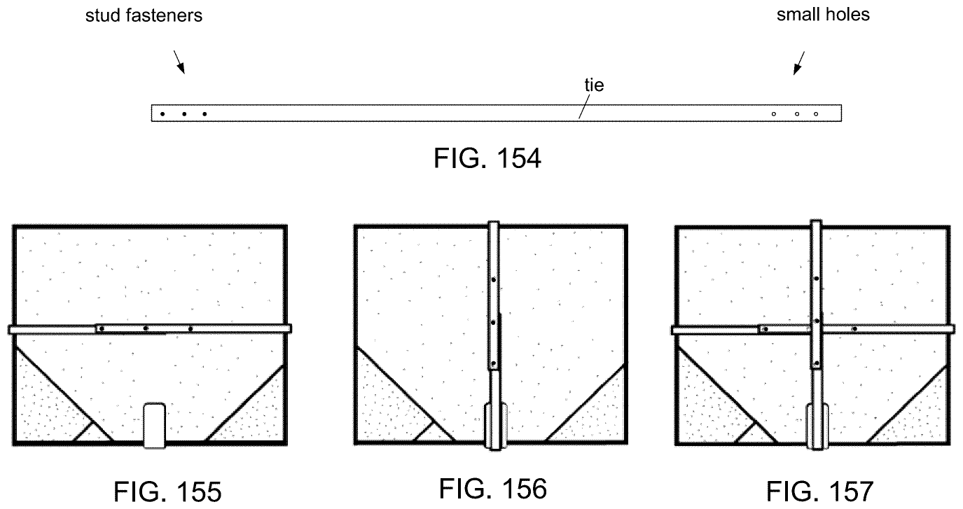

FIG. 154 is a top plan view of another preferred tie that may be used to secure the envelope of FIG. 133, in accordance with one or more aspects and features of the invention.

FIG. 155 is a top plan view of the tie of FIG. 154 used in a first preferred manner to secure the envelope of FIG. 133, in accordance with one or more aspects and features of the invention.

FIG. 156 is a top plan view of the tie of FIG. 154 used in a second preferred manner to secure the envelope of FIG. 133, in accordance with one or more aspects and features of the invention.

FIG. 157 is a top plan view of two ties of FIG. 154 used in a preferred manner to secure the envelope of FIG. 133, in accordance with one or more aspects and features of the invention.

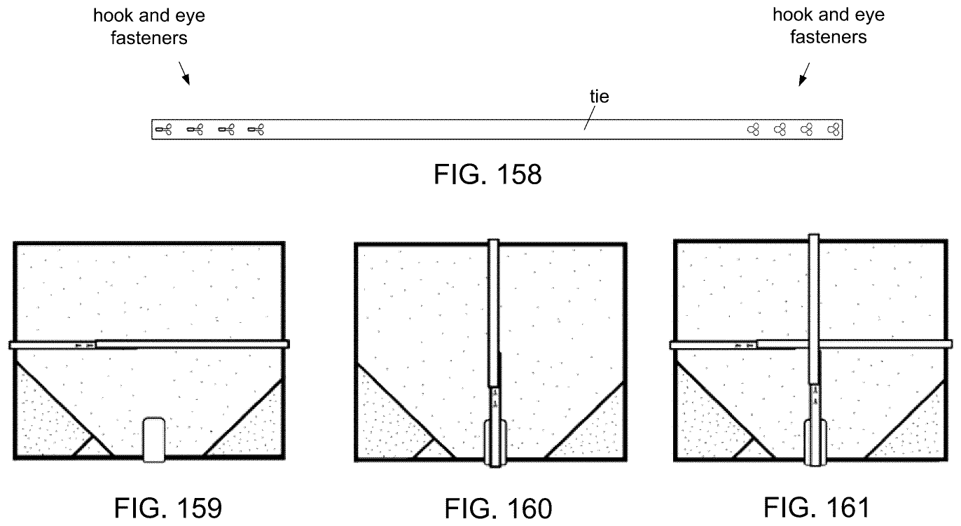

FIG. 158 is a top plan view of another preferred tie that may be used to secure the envelope of FIG. 133, in accordance with one or more aspects and features of the invention.

FIG. 159 is a top plan view of the tie of FIG. 158 used in a first preferred manner to secure the envelope of FIG. 133, in accordance with one or more aspects and features of the invention.

FIG. 160 is a top plan view of the tie of FIG. 158 used in a second preferred manner to secure the envelope of FIG. 133, in accordance with one or more aspects and features of the invention.

FIG. 161 is a top plan view of two ties of FIG. 158 used in a preferred manner to secure the envelope of FIG. 133, in accordance with one or more aspects and features of the invention.

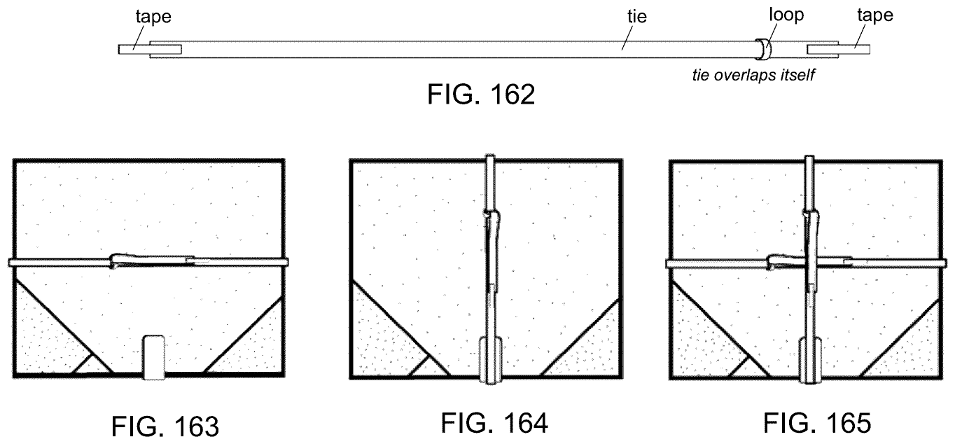

FIG. 162 is a top plan view of another preferred tie that may be used to secure the envelope of FIG. 133, in accordance with one or more aspects and features of the invention.

FIG. 163 is a top plan view of the tie of FIG. 162 used in a first preferred manner to secure the envelope of FIG. 133, in accordance with one or more aspects and features of the invention.

FIG. 164 is a top plan view of the tie of FIG. 162 used in a second preferred manner to secure the envelope of FIG. 133, in accordance with one or more aspects and features of the invention.

FIG. 165 is a top plan view of two ties of FIG. 162 used in a preferred manner to secure the envelope of FIG. 133, in accordance with one or more aspects and features of the invention.

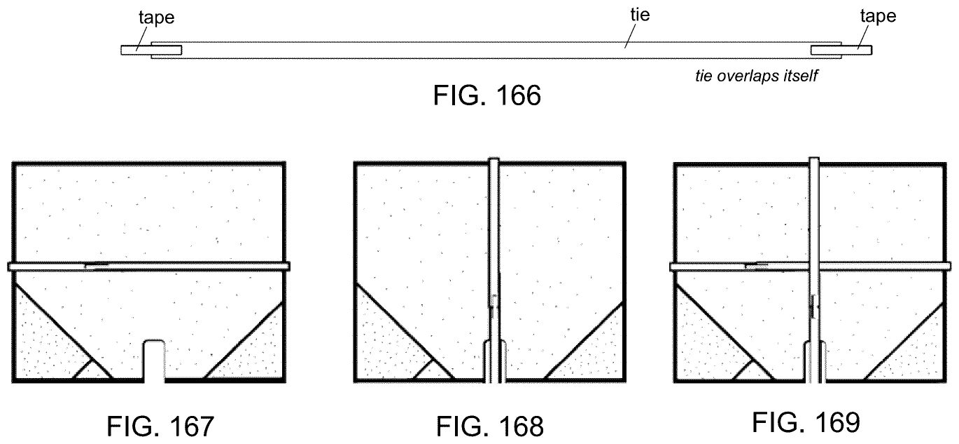

FIG. 166 is a top plan view of another preferred tie that may be used to secure the envelope of FIG. 133, in accordance with one or more aspects and features of the invention.

FIG. 167 is a top plan view of the tie of FIG. 166 used in a first preferred manner to secure the envelope of FIG. 133, in accordance with one or more aspects and features of the invention.

FIG. 168 is a top plan view of the tie of FIG. 166 used in a second preferred manner to secure the envelope of FIG. 133, in accordance with one or more aspects and features of the invention.

FIG. 169 is a top plan view of two ties of FIG. 166 used in a preferred manner to secure the envelope of FIG. 133, in accordance with one or more aspects and features of the invention.

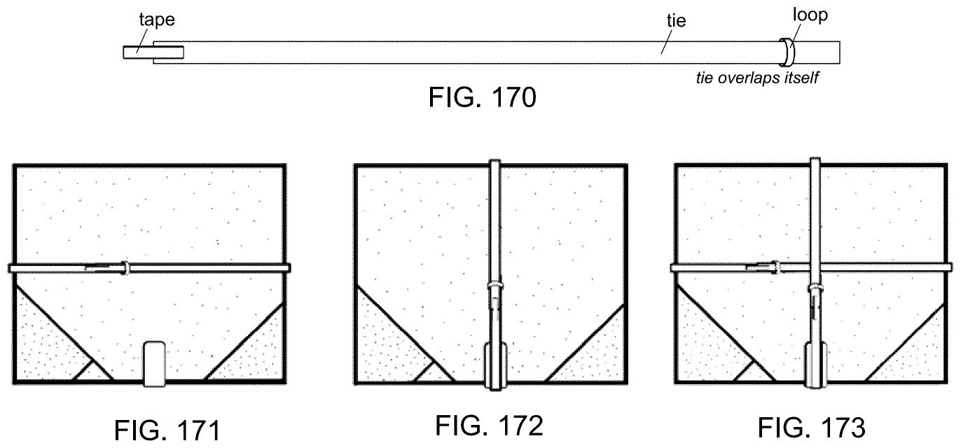

FIG. 170 is a top plan view of another preferred tie that may be used to secure the envelope of FIG. 133, in accordance with one or more aspects and features of the invention.

FIG. 171 is a top plan view of the tie of FIG. 170 used in a first preferred manner to secure the envelope of FIG. 133, in accordance with one or more aspects and features of the invention.

FIG. 172 is a top plan view of the tie of FIG. 170 used in a second preferred manner to secure the envelope of FIG. 133, in accordance with one or more aspects and features of the invention.

FIG. 173 is a top plan view of two ties of FIG. 170 used in a preferred manner to secure the envelope of FIG. 133, in accordance with one or more aspects and features of the invention.

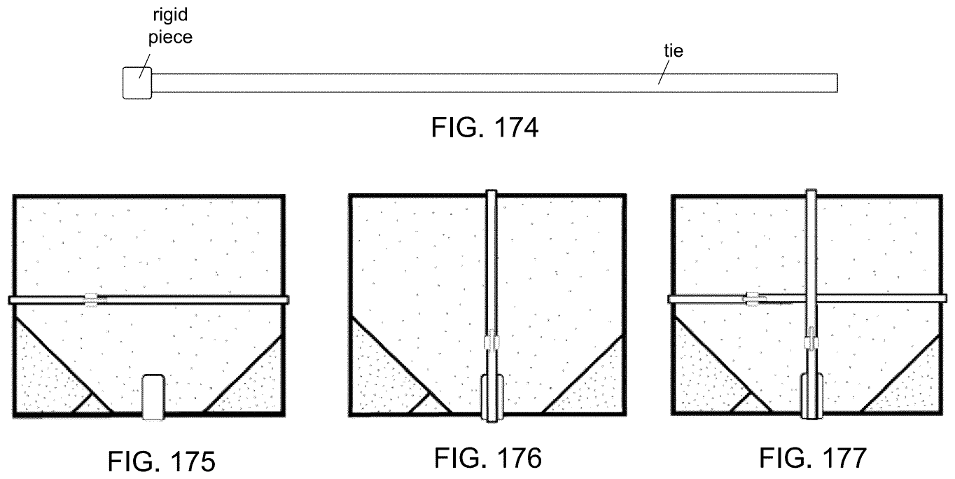

FIG. 174 is a top plan view of another preferred tie that may be used to secure the envelope of FIG. 133, in accordance with one or more aspects and features of the invention.

FIG. 175 is a top plan view of the tie of FIG. 174 used in a first preferred manner to secure the envelope of FIG. 133, in accordance with one or more aspects and features of the invention.

FIG. 176 is a top plan view of the tie of FIG. 174 used in a second preferred manner to secure the envelope of FIG. 133, in accordance with one or more aspects and features of the invention.

FIG. 177 is a top plan view of two ties of FIG. 174 used in a preferred manner to secure the envelope of FIG. 133, in accordance with one or more aspects and features of the invention.

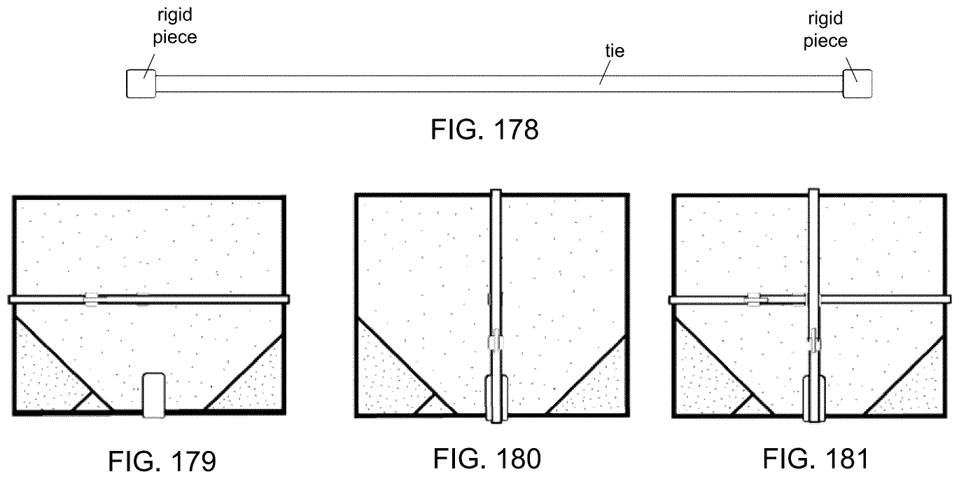

FIG. 178 is a top plan view of another preferred tie that may be used to secure the envelope of FIG. 133, in accordance with one or more aspects and features of the invention.

FIG. 179 is a top plan view of the tie of FIG. 178 used in a first preferred manner to secure the envelope of FIG. 133, in accordance with one or more aspects and features of the invention.

FIG. 180 is a top plan view of the tie of FIG. 178 used in a second preferred manner to secure the envelope of FIG. 133, in accordance with one or more aspects and features of the invention.

FIG. 181 is a top plan view of two ties of FIG. 178 used in a preferred manner to secure the envelope of FIG. 133, in accordance with one or more aspects and features of the invention.

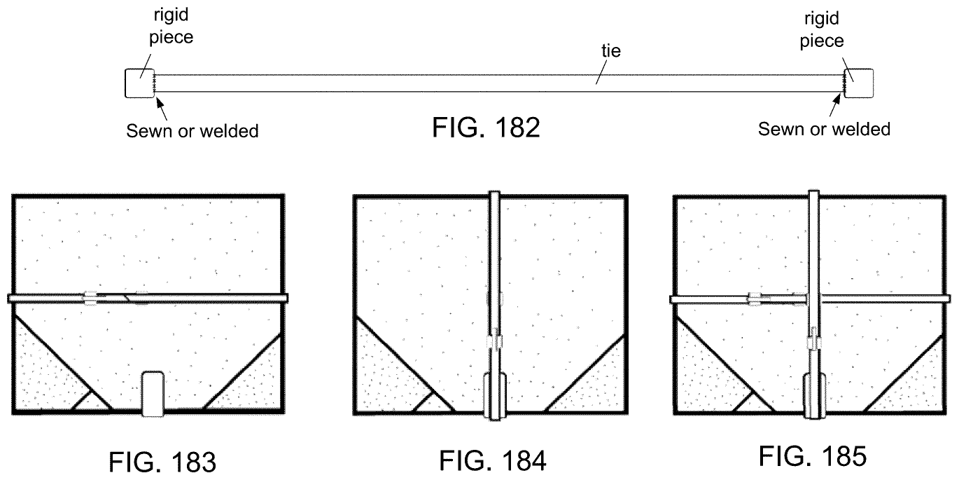

FIG. 182 is a top plan view of another preferred tie that may be used to secure the envelope of FIG. 133, in accordance with one or more aspects and features of the invention.

FIG. 183 is a top plan view of the tie of FIG. 182 used in a first preferred manner to secure the envelope of FIG. 133, in accordance with one or more aspects and features of the invention.

FIG. 184 is a top plan view of the tie of FIG. 182 used in a second preferred manner to secure the envelope of FIG. 133, in accordance with one or more aspects and features of the invention.

FIG. 185 is a top plan view of two ties of FIG. 182 used in a preferred manner to secure the envelope of FIG. 133, in accordance with one or more aspects and features of the invention.

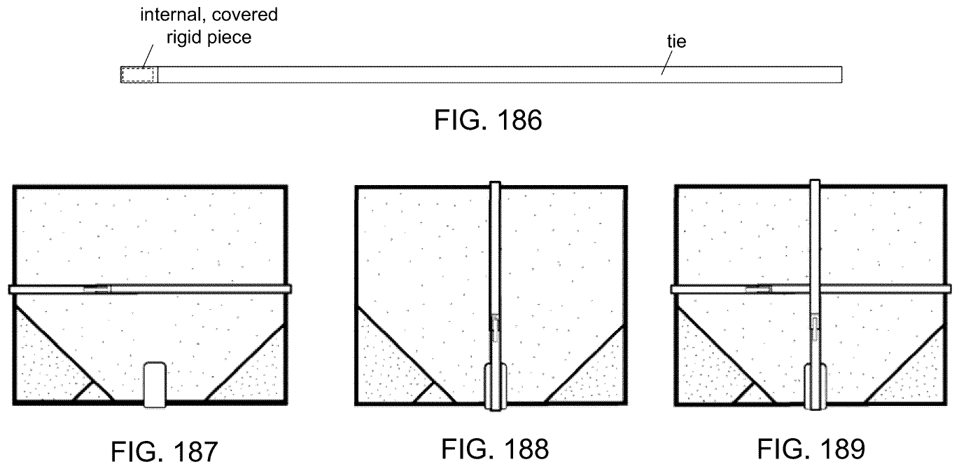

FIG. 186 is a top plan view of another preferred tie that may be used to secure the envelope of FIG. 133, in accordance with one or more aspects and features of the invention.

FIG. 187 is a top plan view of the tie of FIG. 186 used in a first preferred manner to secure the envelope of FIG. 133, in accordance with one or more aspects and features of the invention.

FIG. 188 is a top plan view of the tie of FIG. 186 used in a second preferred manner to secure the envelope of FIG. 133, in accordance with one or more aspects and features of the invention.

FIG. 189 is a top plan view of two ties of FIG. 186 used in a preferred manner to secure the envelope of FIG. 133, in accordance with one or more aspects and features of the invention.

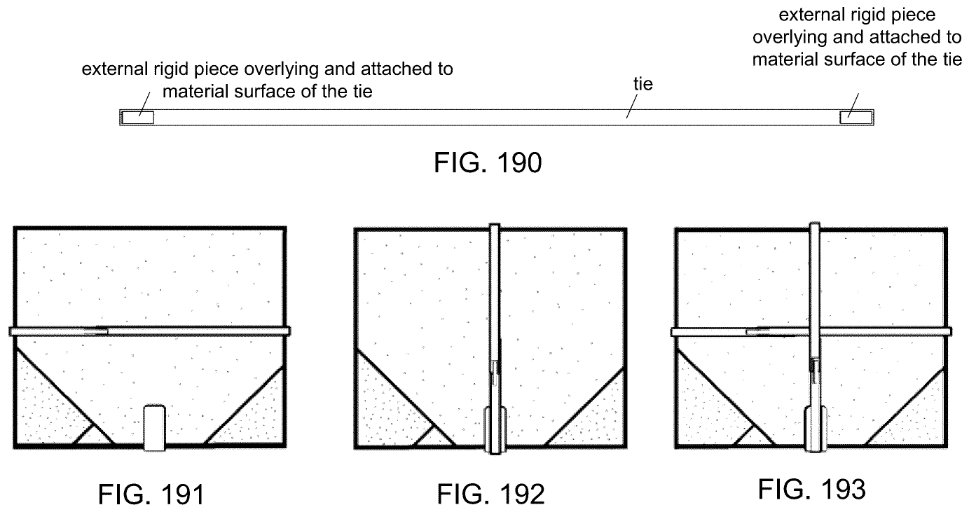

FIG. 190 is a top plan view of another preferred tie that may be used to secure the envelope of FIG. 133, in accordance with one or more aspects and features of the invention.

FIG. 191 is a top plan view of the tie of FIG. 190 used in a first preferred manner to secure the envelope of FIG. 133, in accordance with one or more aspects and features of the invention.

FIG. 192 is a top plan view of the tie of FIG. 190 used in a second preferred manner to secure the envelope of FIG. 133, in accordance with one or more aspects and features of the invention.

FIG. 193 is a top plan view of two ties of FIG. 190 used in a preferred manner to secure the envelope of FIG. 133, in accordance with one or more aspects and features of the invention.

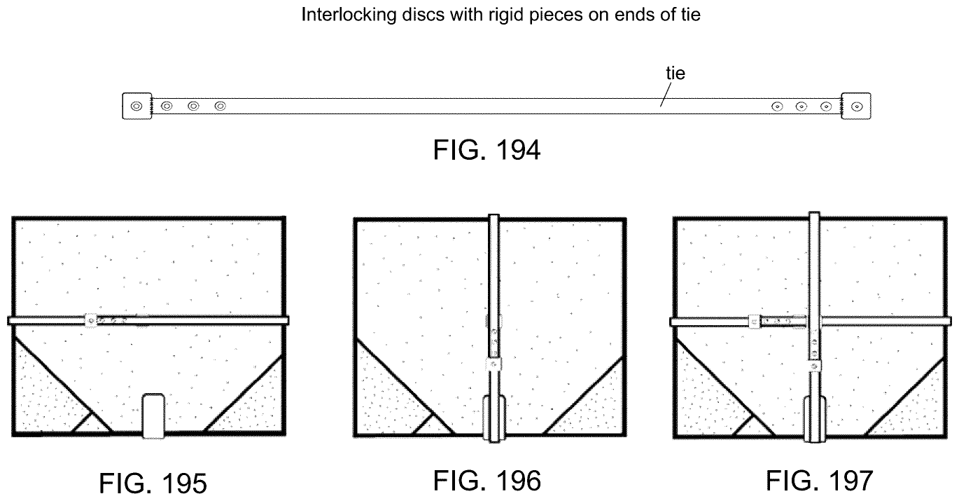

FIG. 194 is a top plan view of another preferred tie that may be used to secure the envelope of FIG. 133, in accordance with one or more aspects and features of the invention.

FIG. 195 is a top plan view of the tie of FIG. 194 used in a first preferred manner to secure the envelope of FIG. 133, in accordance with one or more aspects and features of the invention.

FIG. 196 is a top plan view of the tie of FIG. 194 used in a second preferred manner to secure the envelope of FIG. 133, in accordance with one or more aspects and features of the invention.

FIG. 197 is a top plan view of two ties of FIG. 194 used in a preferred manner to secure the envelope of FIG. 133, in accordance with one or more aspects and features of the invention.

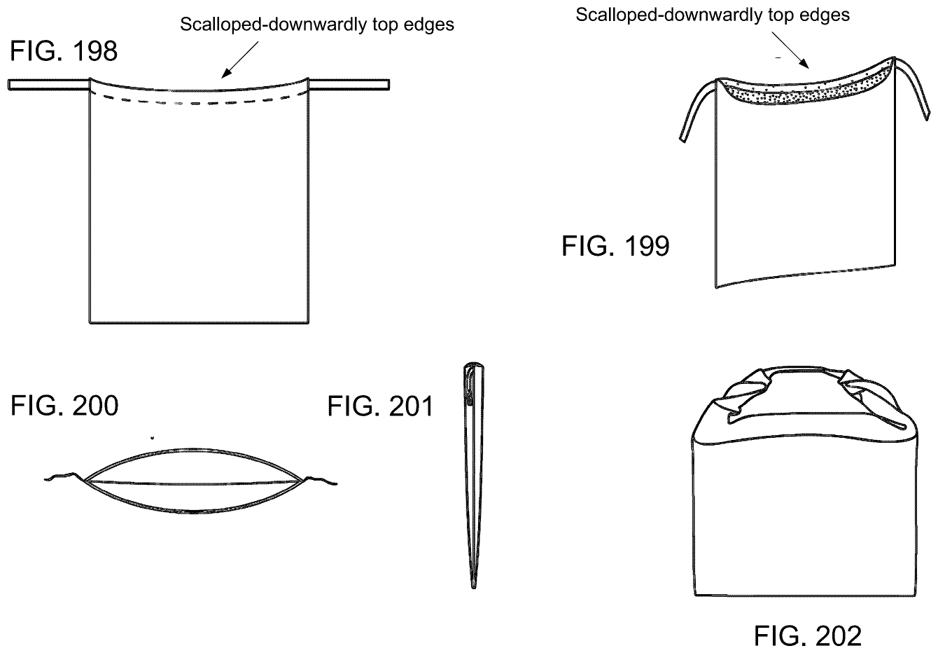

FIG. 198 is a front elevational view of another bag of a preferred embodiment of a method in accordance with one or more aspects and features of the invention.

FIG. 199 is a perspective view of the bag of FIG. 198.

FIG. 200 is a top plan view of the bag of FIG. 198.

FIG. 201 is a side plan view of the bag of FIG. 198.

FIG. 202 is a front elevational view of the bag of FIG. 198 secured in the closed configuration.

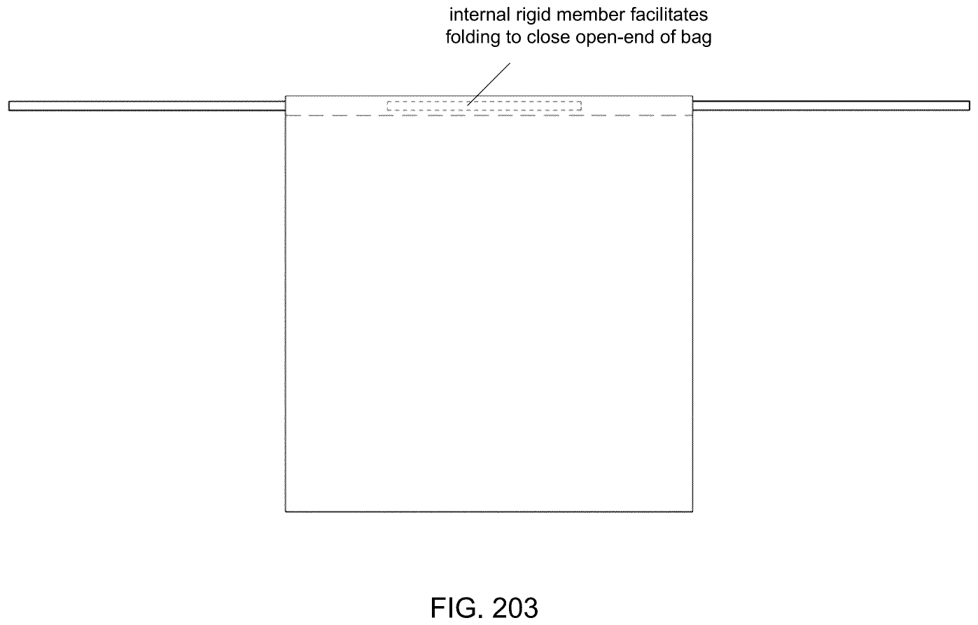

FIG. 203 is a front elevational view of another preferred bag in accordance with one or more aspects and features of the invention.

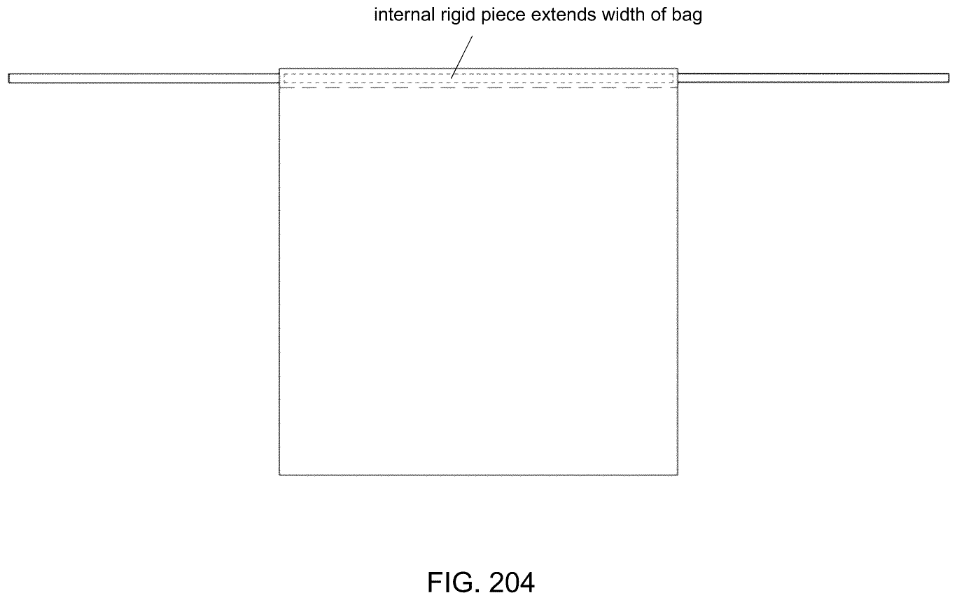

FIG. 204 is a front elevational view of another preferred bag in accordance with one or more aspects and features of the invention.

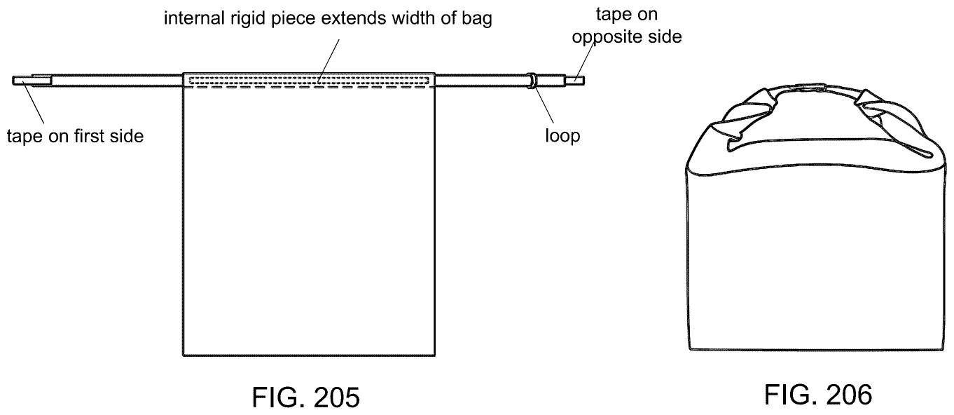

FIG. 205 is a front elevational view of another preferred bag in accordance with one or more aspects and features of the invention.

FIG. 206 is a front elevational view of the bag of FIG. 205 secured in the closed configuration.

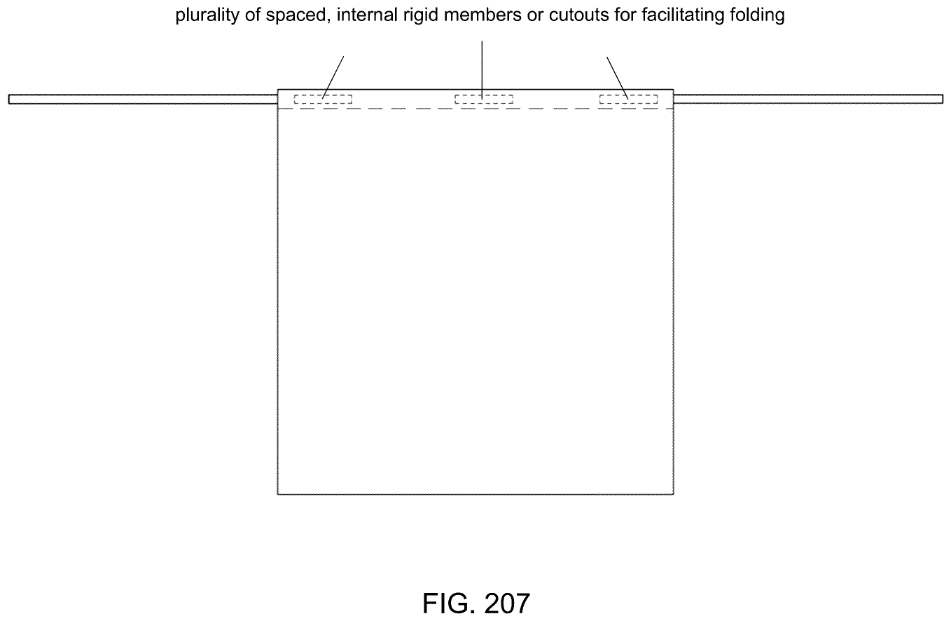

FIG. 207 is a front elevational view of another preferred bag in accordance with one or more aspects and features of the invention.

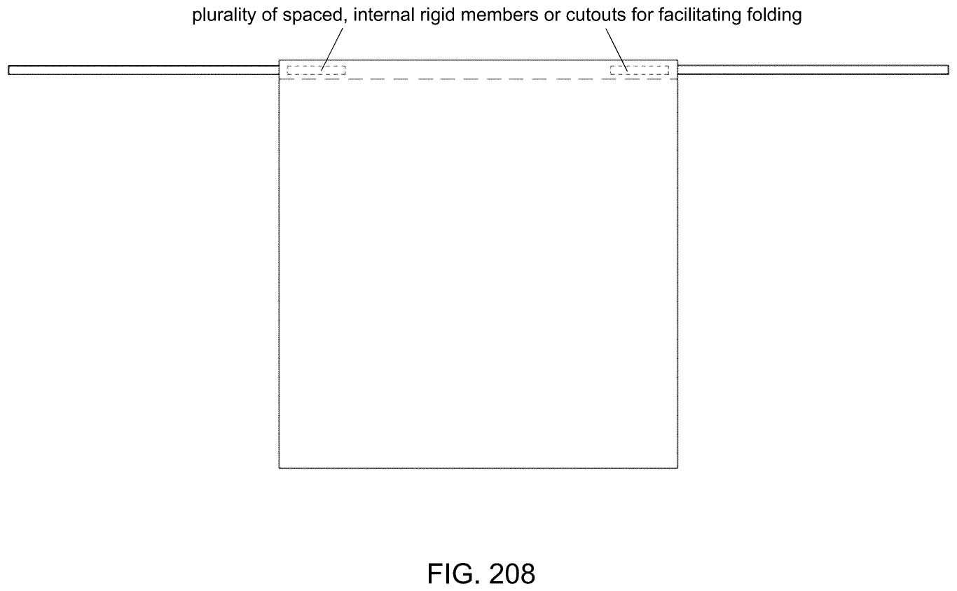

FIG. 208 is a front elevational view of another preferred bag in accordance with one or more aspects and features of the invention.

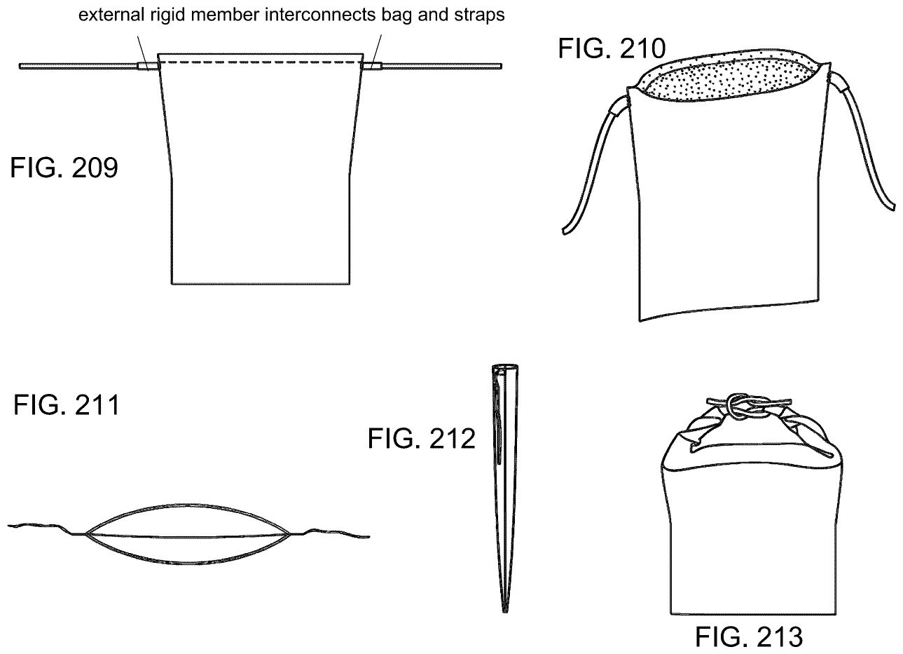

FIG. 209 is a front elevational view of another bag of a preferred embodiment of a method in accordance with one or more aspects and features of the invention.

FIG. 210 is a perspective view of the bag of FIG. 209.

FIG. 211 is a top plan view of the bag of FIG. 209.

FIG. 212 is a side plan view of the bag of FIG. 209.

FIG. 213 is a front elevational view of the bag of FIG. 209 secured in the closed configuration.

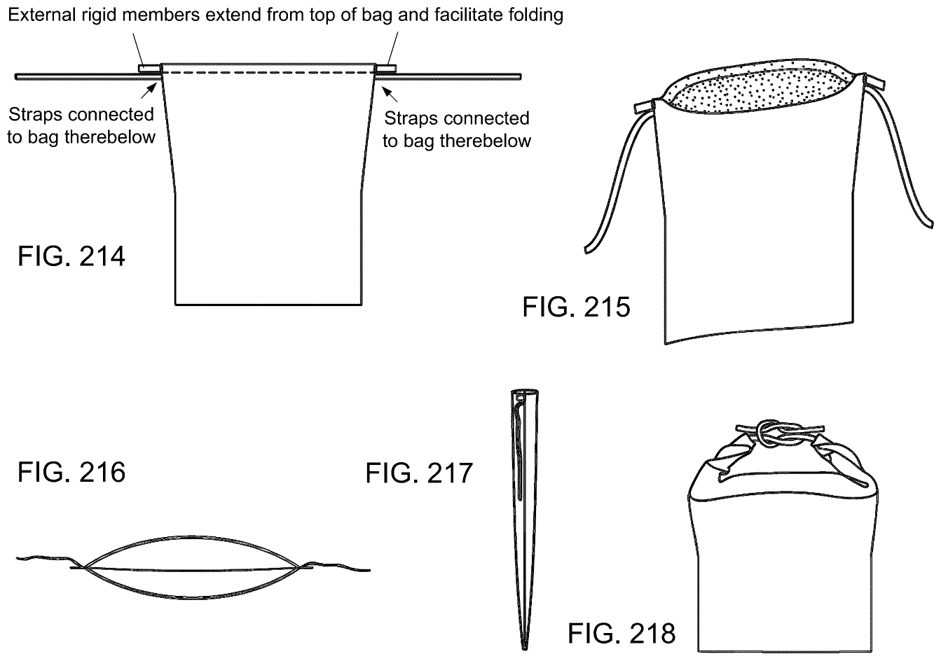

FIG. 214 is a front elevational view of another bag of a preferred embodiment of a method in accordance with one or more aspects and features of the invention.

FIG. 215 is a perspective view of the bag of FIG. 214.

FIG. 216 is a top plan view of the bag of FIG. 214.

FIG. 217 is a side plan view of the bag of FIG. 214.

FIG. 218 is a front elevational view of the bag of FIG. 214 secured in the closed configuration.

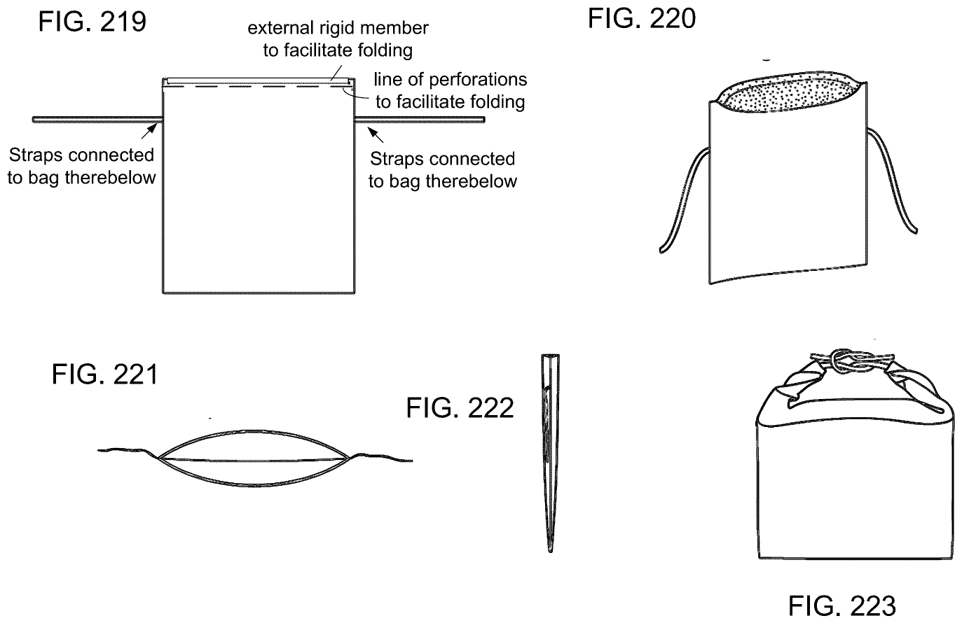

FIG. 219 is a front elevational view of another bag of a preferred embodiment of a method in accordance with one or more aspects and features of the invention.

FIG. 220 is a perspective view of the bag of FIG. 219.

FIG. 221 is a top plan view of the bag of FIG. 219.

FIG. 222 is a side plan view of the bag of FIG. 219.

FIG. 223 is a front elevational view of the bag of FIG. 219 secured in the closed configuration.

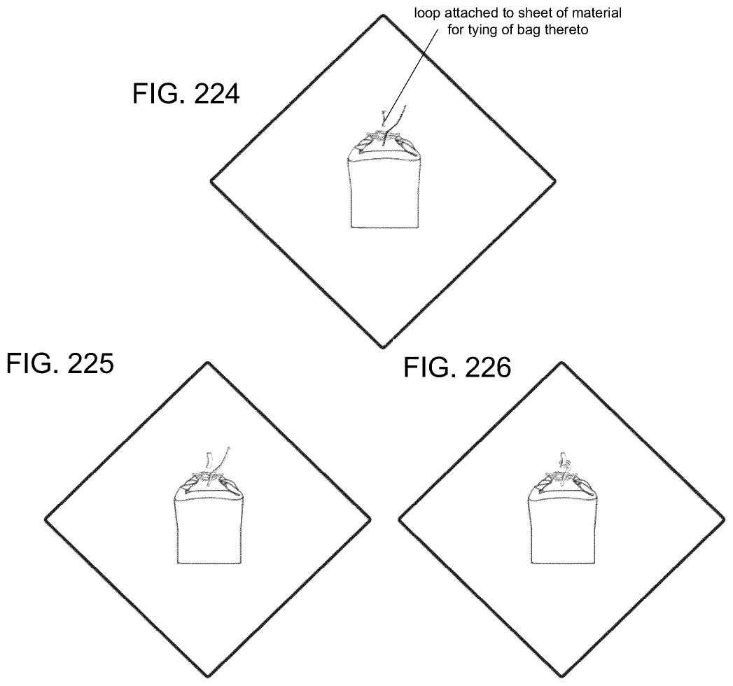

FIGS. 224-226 illustrate preliminary steps of a preferred method of enveloping a bag secured in a closed configuration by folding a sheet around the bag to entirely envelope the bag, in accordance with one or more aspects and features of the invention.

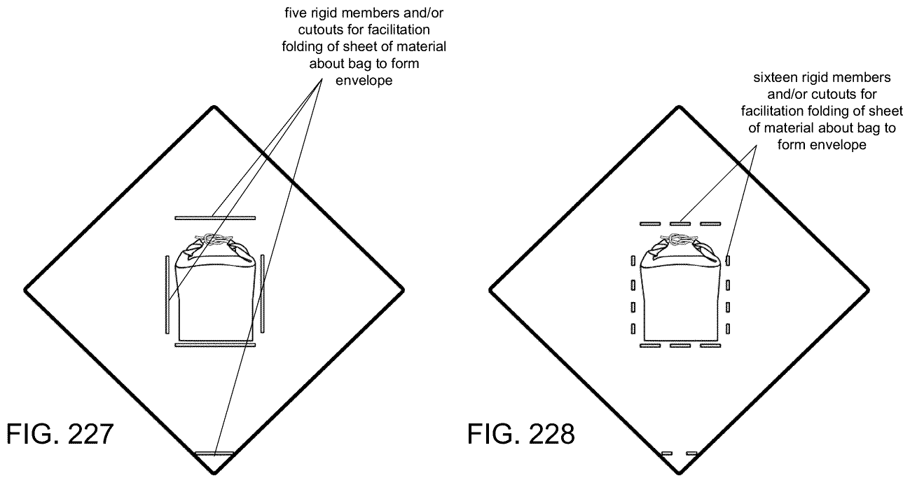

FIG. 227 illustrates an alternative sheet of material used in enveloping a bag secured in a closed configuration, in accordance with one or more aspects and features of the invention.

FIG. 228 illustrates an alternative sheet of material used in enveloping a bag secured in a closed configuration, in accordance with one or more aspects and features of the invention.

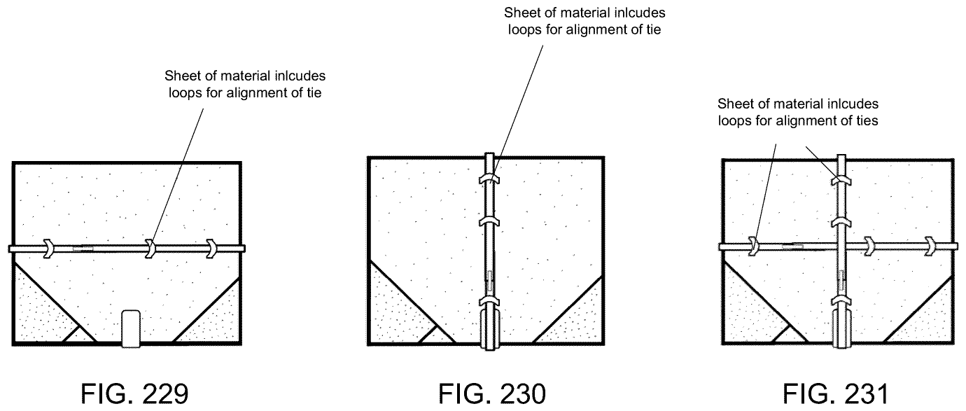

FIG. 229 is a top plan view of another preferred embodiment of an envelope secured about a bag in accordance with one or more aspects and features of the invention.

FIG. 230 is a top plan view of another preferred embodiment of an envelope secured about a bag in accordance with one or more aspects and features of the invention.

FIG. 231 is a top plan view of another preferred embodiment of an envelope secured about a bag in accordance with one or more aspects and features of the invention.

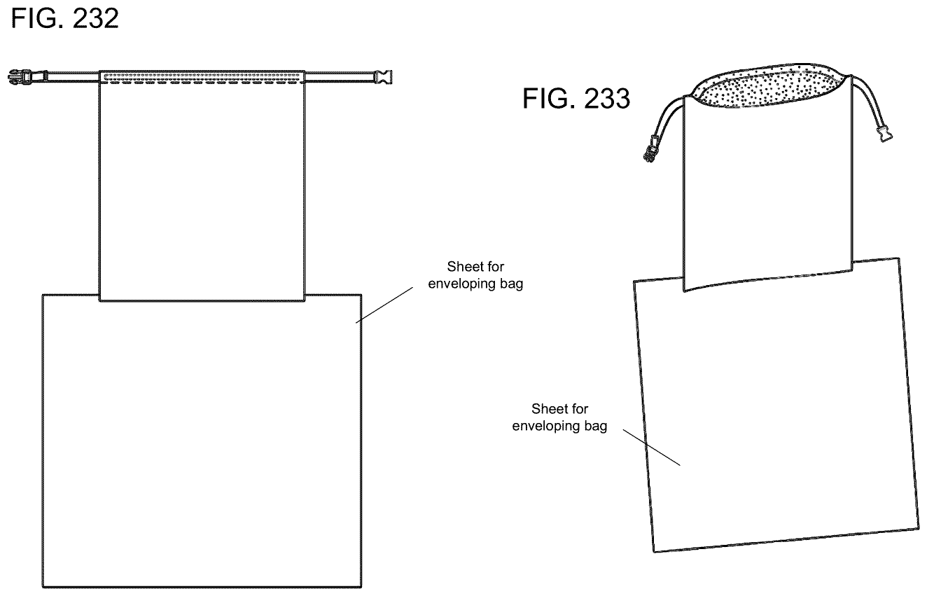

FIG. 232 is a front elevational view of another preferred embodiment of a bag in accordance with one or more aspects and features of the invention.

FIG. 233 is a perspective view of the bag of FIG. 232.

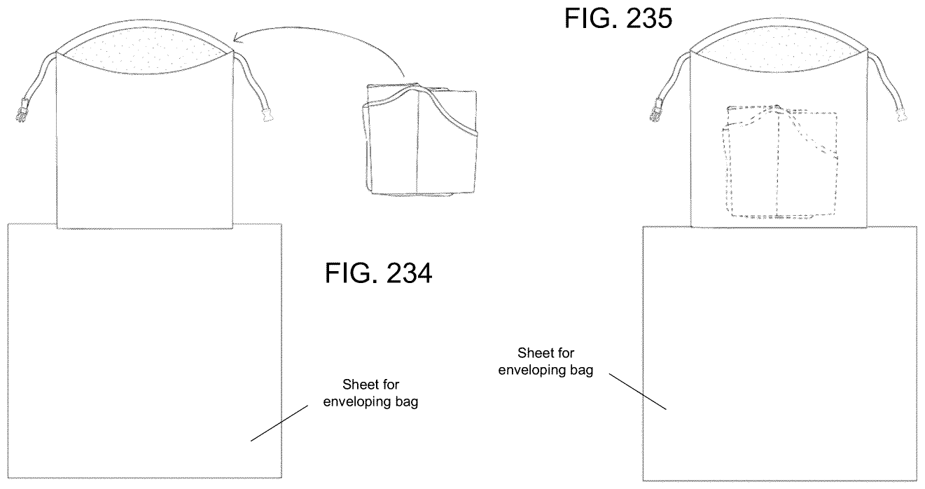

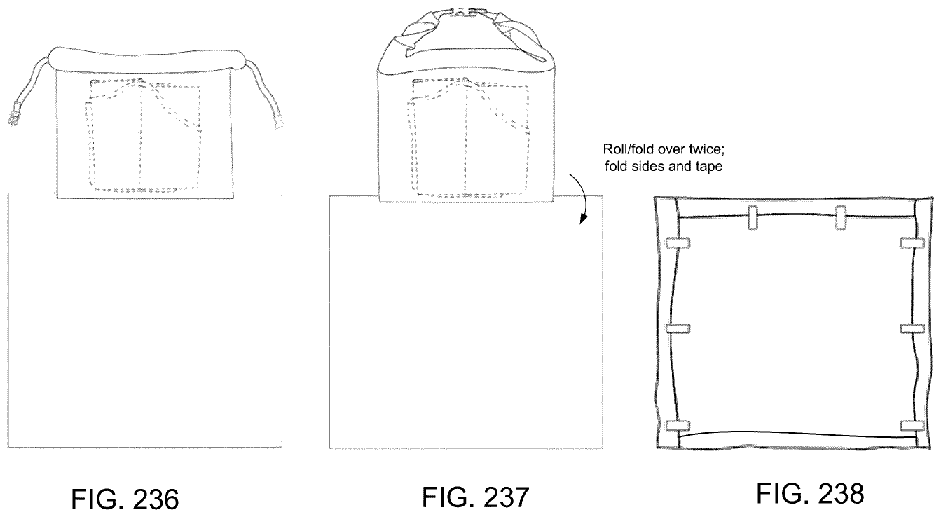

FIGS. 234-238 illustrate steps of another preferred method of packaging a textile item using the bag of FIG. 232, in accordance with one or more aspects and features of the invention.

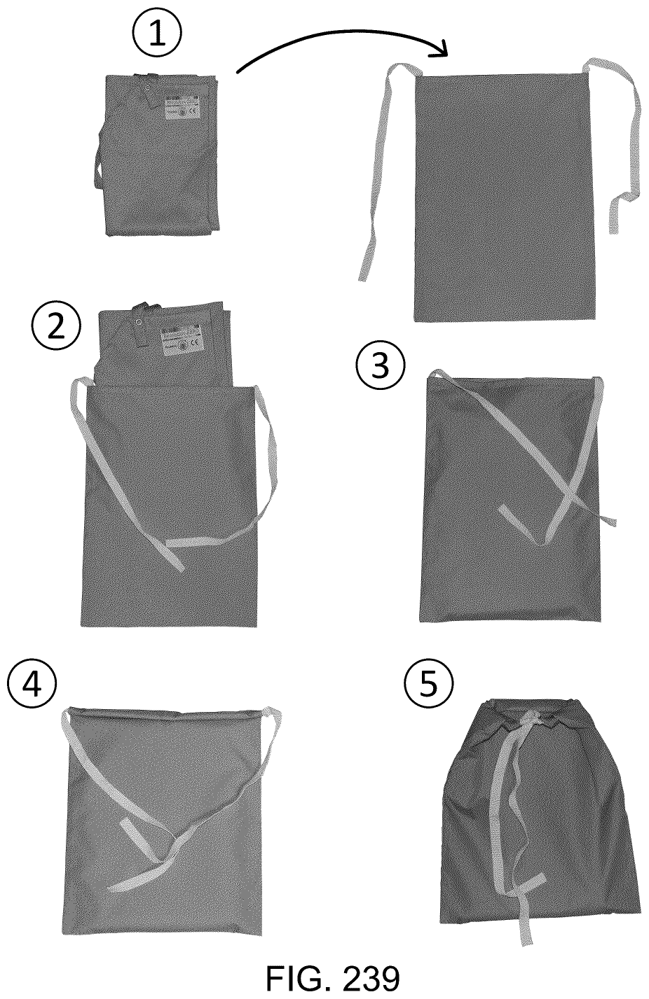

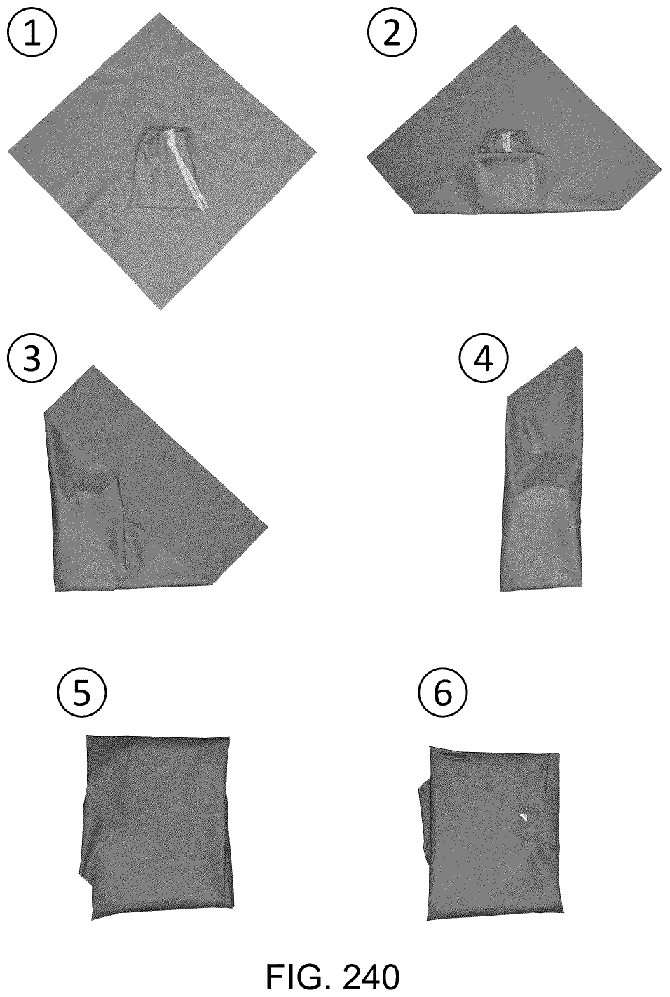

FIGS. 239-240 illustrate a first sequence of illustrations 1-5 in which a textile item is inserted and secured within a bag, and a subsequent sequence of illustrations 1-6 in which the bag in turn is enveloped and secured within a sheet, all for packaging a textile item in accordance with one or more aspects and features of the invention. The resulting package preferably constitutes mono material packaging.

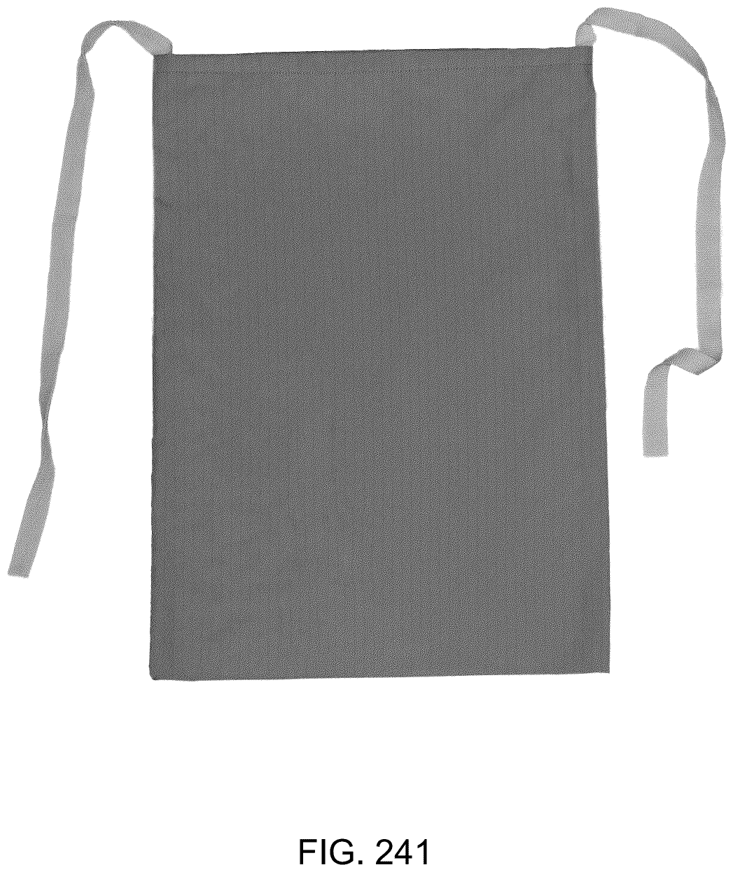

FIG. 241 illustrates part of the first sequence of FIG. 239 and, in particular, a part of illustration 1 of FIG. 239 showing the bag that is used.

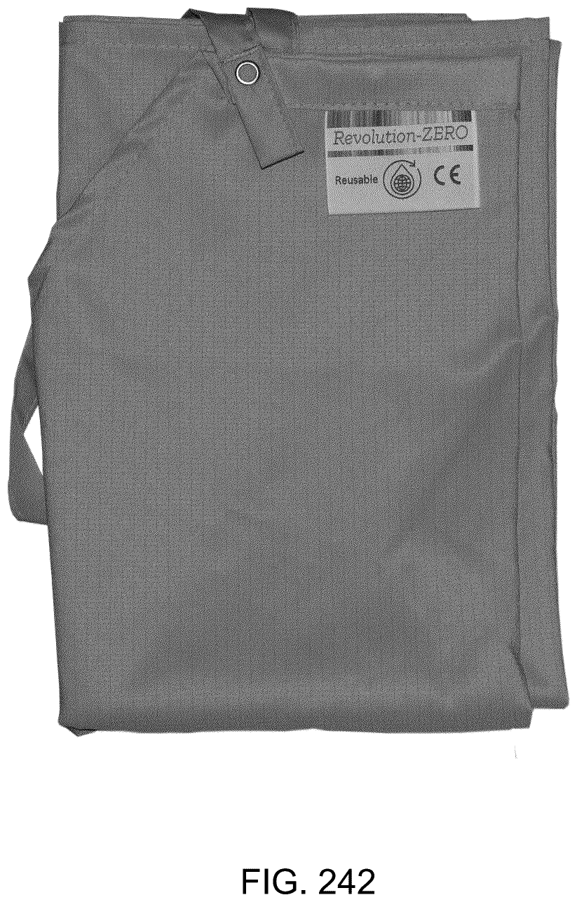

FIG. 242 illustrates another part of the first sequence of FIG. 239 and, in particular, a part of illustration 1 showing a textile item which is the subject of the packaging of the sequence.

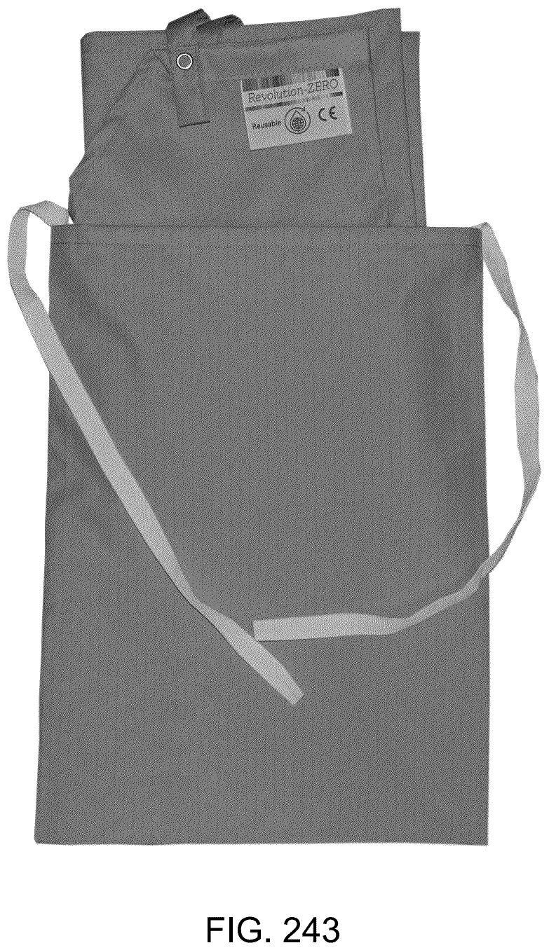

FIG. 243 illustrates another part of the first sequence of FIG. 239 and, in particular, illustration 2 of FIG. 239 showing the insertion of the textile item of FIG. 242 into the bag of FIG. 241.

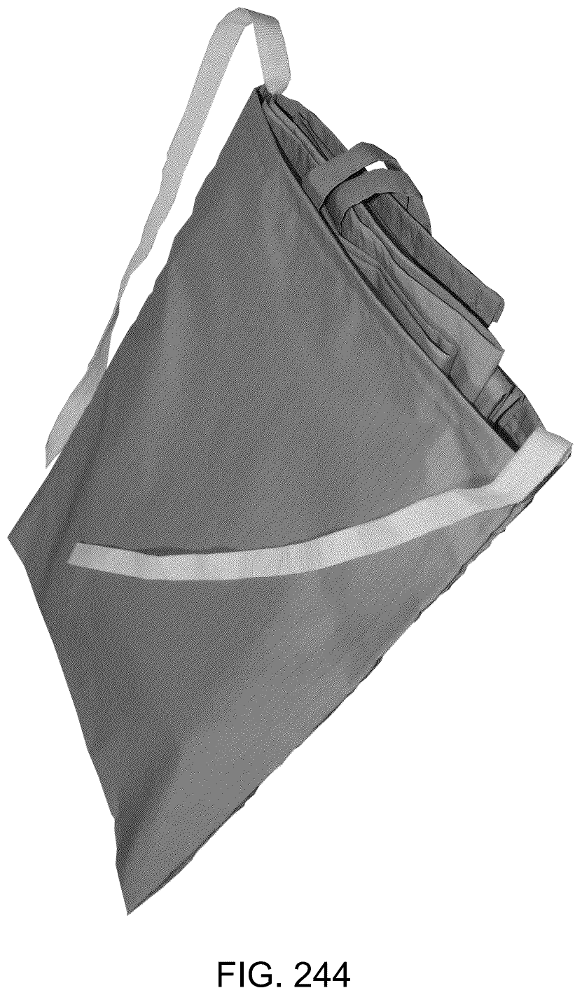

FIG. 244 further illustrates the insertion of the textile item of FIG. 242 into the bag of FIG. 241.

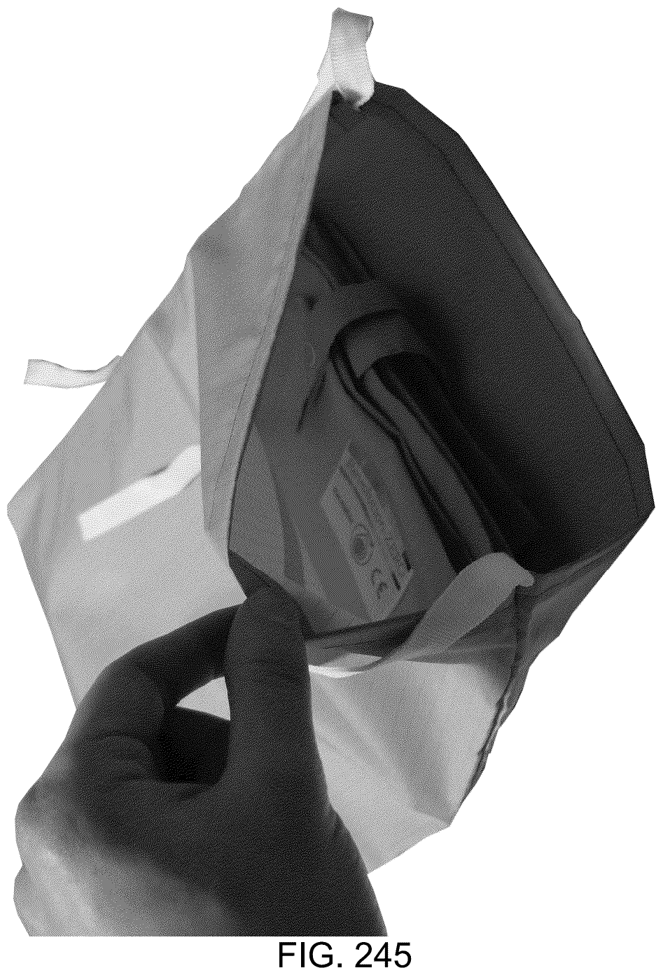

FIG. 245 illustrates the textile item of FIG. 242 having been inserted into the bag of FIG. 241.

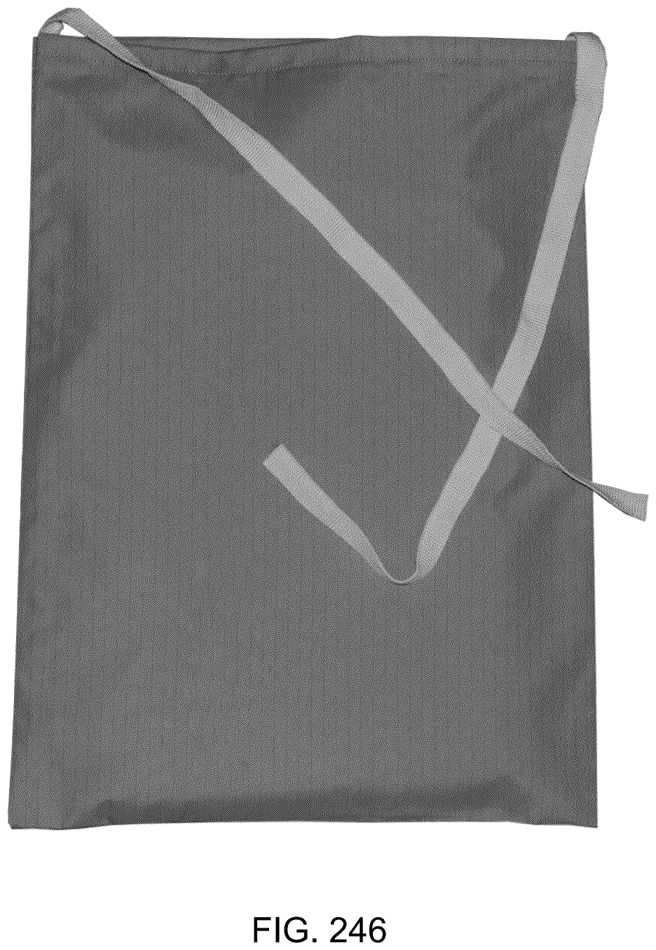

FIG. 246 illustrates further the textile item of FIG. 242 having been inserted into the bag of FIG. 241.

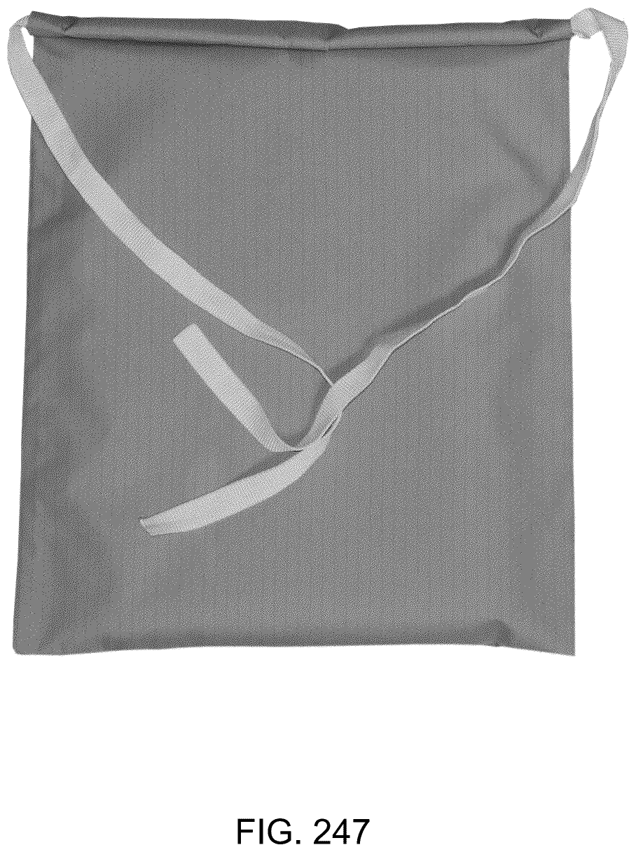

FIG. 247 illustrates another part of the first sequence of FIG. 239 and, in particular, illustration 3 of FIG. 239 showing the rolling closed of the open end of the bag of FIG. 246.

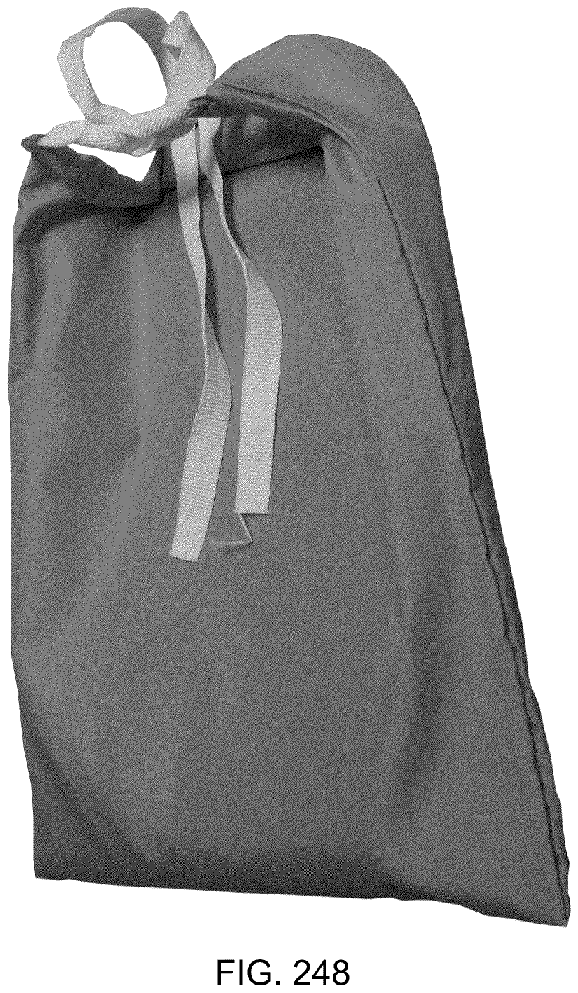

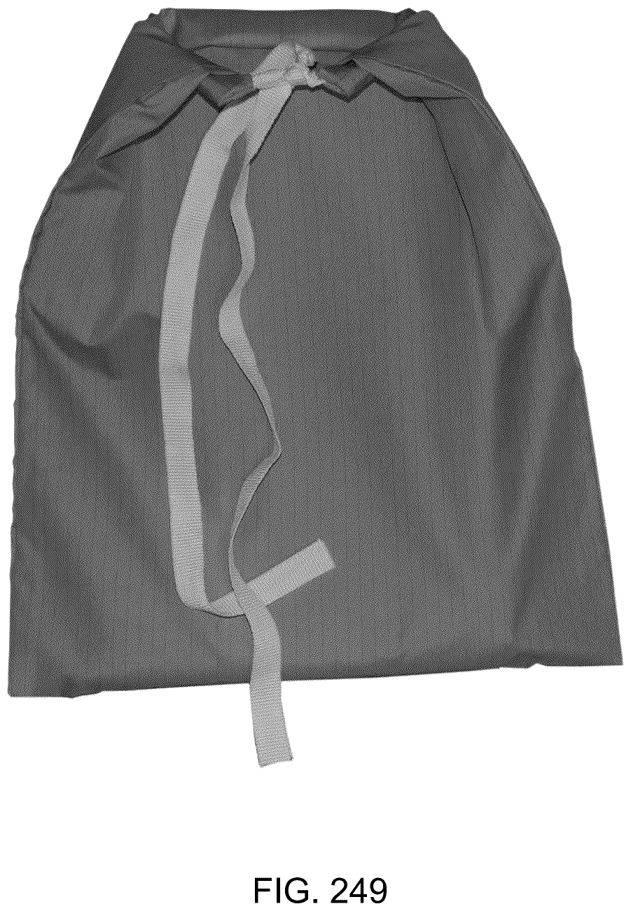

FIG. 248 illustrates another part of the first sequence of FIG. 239 and, in particular, illustration 4 of FIG. 239 showing the securing of the closed end of the bag of FIG. 247 by tying.

FIG. 249 illustrates another part of the first sequence of FIG. 239 and, in particular, illustration 5 of FIG. 239 showing the bag of FIG. 248 secured in a closed configuration and ready for the subsequent sequence.

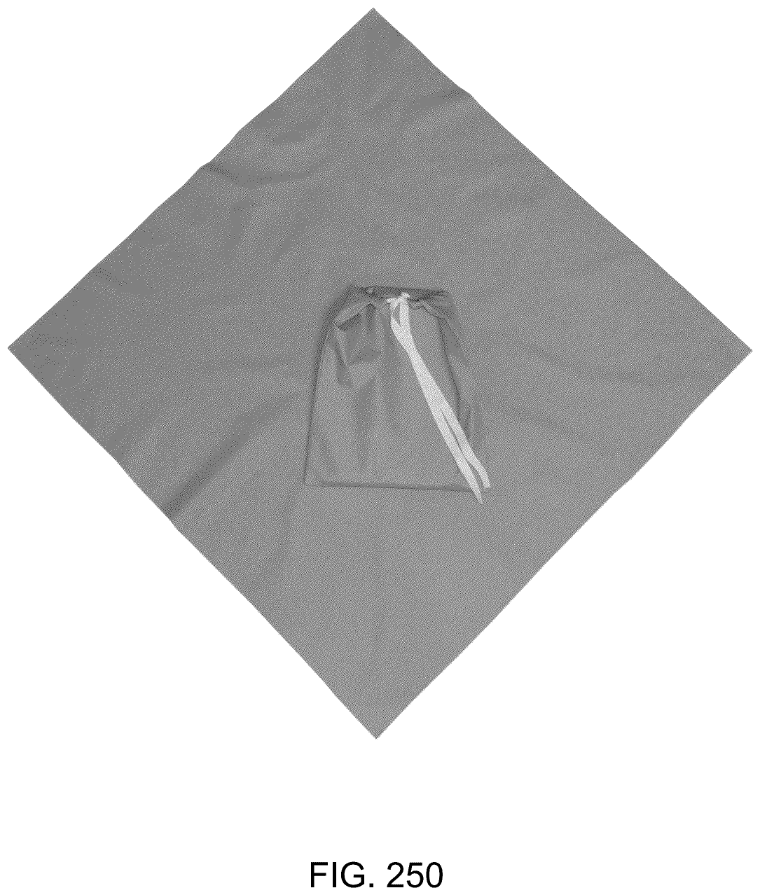

FIG. 250 illustrates part of the subsequent sequence of FIG. 240 and, in particular, illustration 1 of FIG. 240 showing the placement of the bag of FIG. 249 onto a sheet.

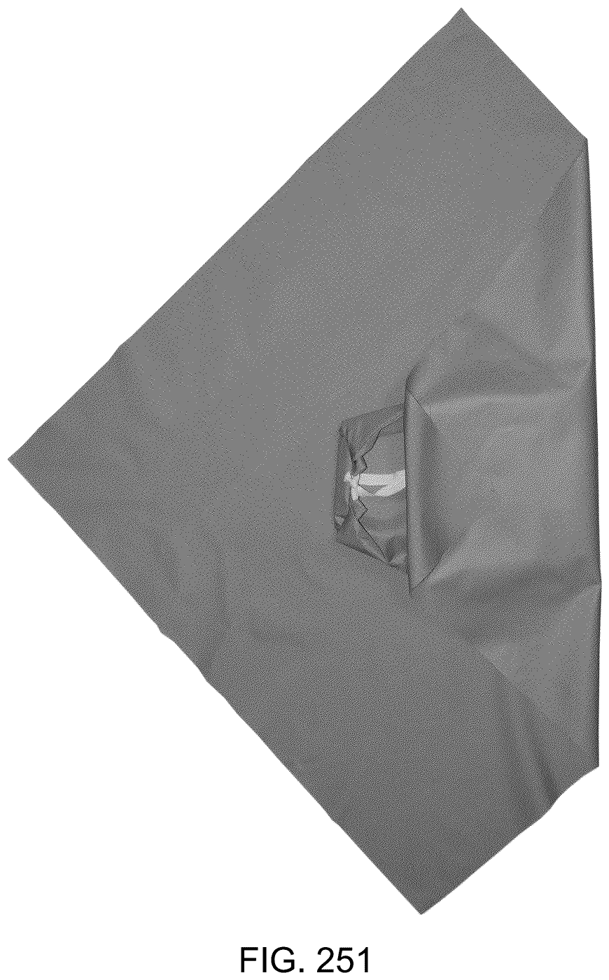

FIG. 251 illustrates another part of the subsequent sequence of FIG. 240 and, in particular, illustration 2 of FIG. 240 showing folding of the sheet about the bag shown in FIG. 250.

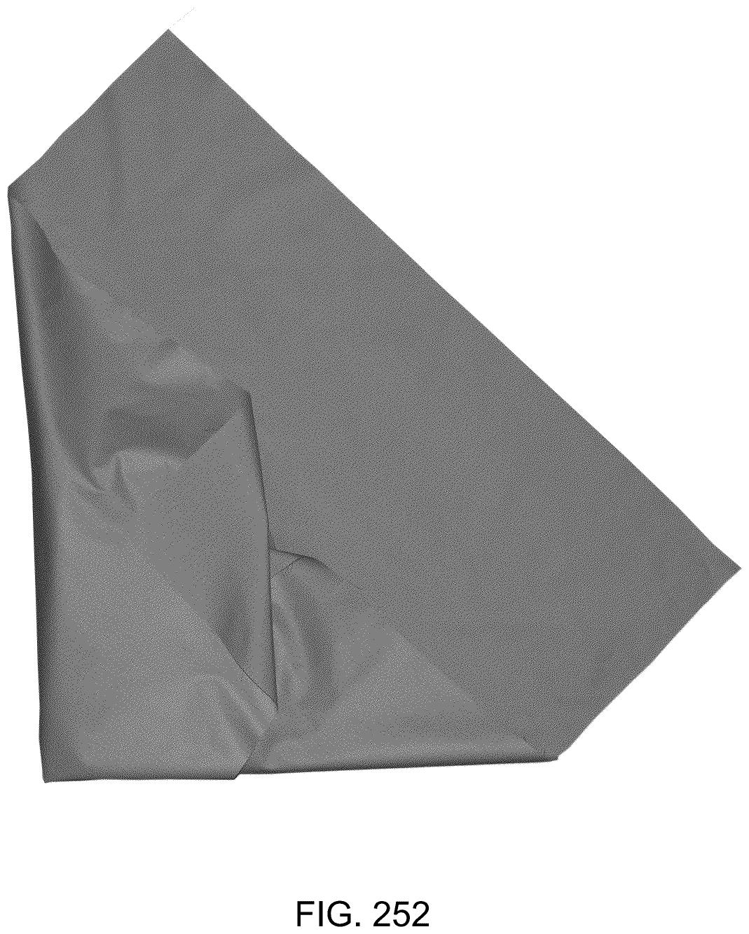

FIG. 252 illustrates another part of the subsequent sequence of FIG. 240 and, in particular, illustration 3 of FIG. 240 showing further folding of the sheet about the bag shown in FIG. 251.

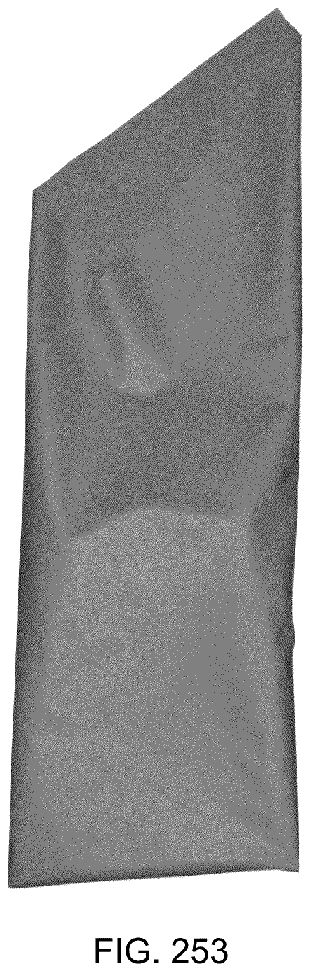

FIG. 253 illustrates another part of the subsequent sequence of FIG. 240 and, in particular, illustration 4 of FIG. 240 showing further folding of the sheet about the bag shown in FIG. 252.

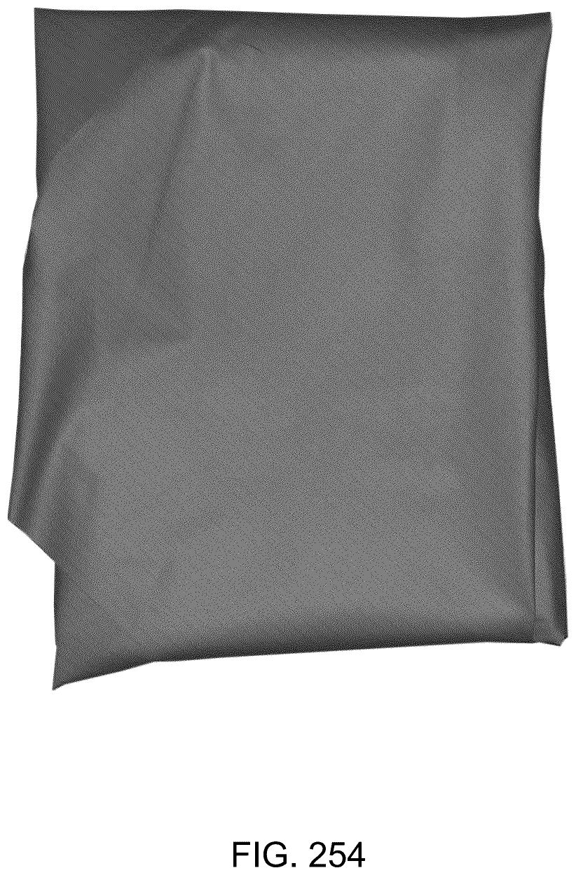

FIG. 254 illustrates another part of the subsequent sequence of FIG. 240 and, in particular, illustration 5 of FIG. 240 showing further folding of the sheet about the bag shown in FIG. 253.

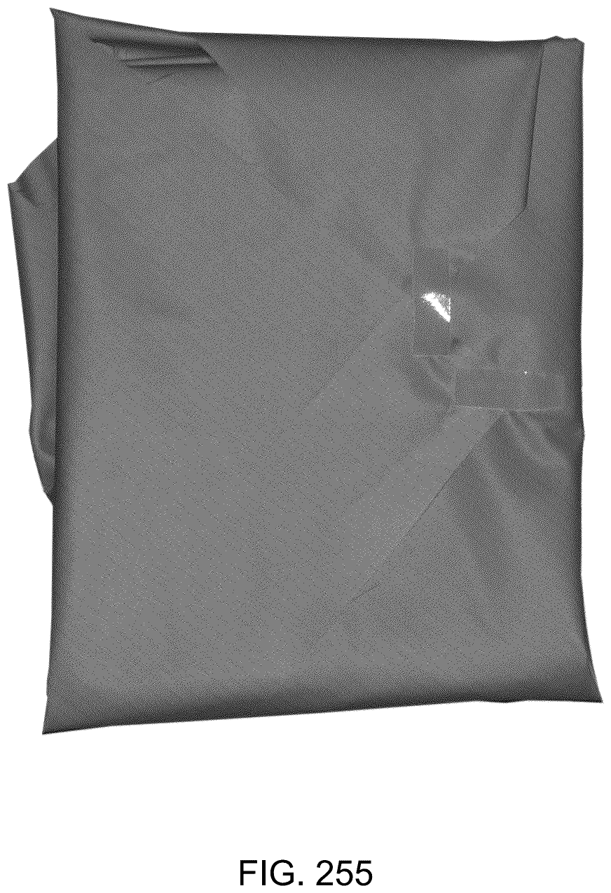

FIG. 255 illustrates another part of the subsequent sequence of FIG. 240 and, in particular, illustration 6 of FIG. 250 showing the securing of the envelope folded about the shown in FIG. 254 using tape and, preferably, dissolvable tape.

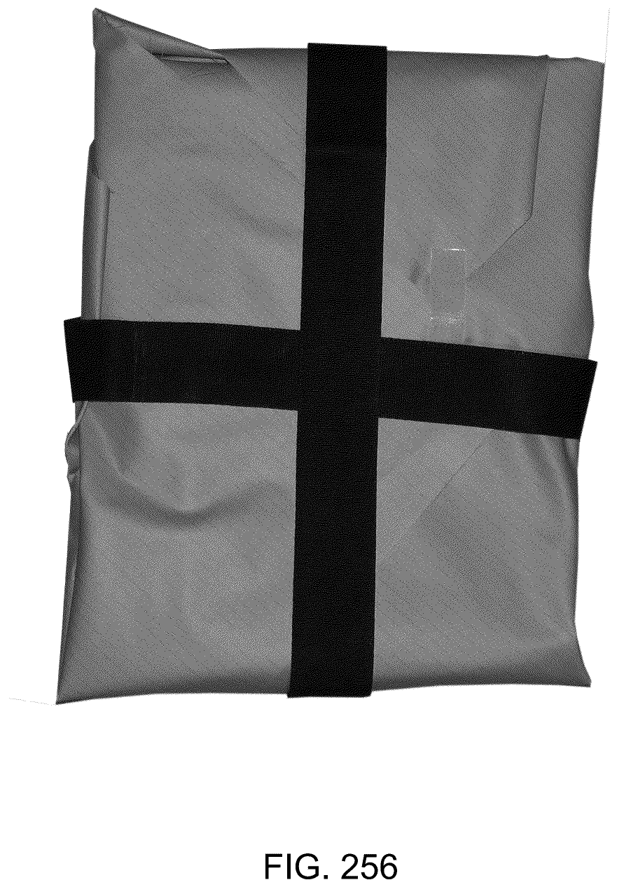

FIG. 256 illustrates a final, optional step of securing the envelope around the bag shown in FIG. 255 using ties.

In accordance with one or more aspects and features of the invention, the packaged textile item is labelled. If a label card is to be used it would ideally be made of polyester with a printed label put on this, which would be secured on the outside of the envelope.

In accordance with one or more aspects and features of the invention, the packaged textile item is sterilized for use in a sterile environment.

When the packaged textile item is to be used, any external tie is undone, and the folded sheet is unfolded and laid opened in order to provide a sterile field for placing the textile item onto. The fastening system for the internal envelope is undone and the user picks up the pouch and shakes out the textile item onto the sterile field created by the inside section of the outer sheet that defined the envelope. The user then places the bag to the side for cleaning and uses the textile item as they normal would in the sterile environment.

As will be appreciated from the foregoing description, embodiments in accordance with one or more aspects and features of the invention relate to packaging systems that are designed: to be highly effective in the protection and preservation of an aseptic environment for medical textiles during transport, storage and use; to integrate within clinical pathways to save time and resource in both use and reprocessing; and to utilize materials that are designed for reusing rather than discarding or recycling. Such preferred embodiments comprise methods, apparatus, and systems in which medical textiles are packaged so as to protect and preserve cleanliness and sterility as well as enable ease of use and reprocessing.

Based on the foregoing description, it will be readily understood by those persons skilled in the art that the invention has broad utility and application. Many embodiments and adaptations of the invention other than those specifically described herein, as well as many variations, modifications, and equivalent arrangements, will be apparent from or reasonably suggested by the invention and the foregoing descriptions thereof, without departing from the substance or scope of the invention.

Accordingly, while the invention has been described herein in detail in relation to one or more preferred embodiments, it is to be understood that this disclosure is only illustrative and exemplary of the invention and is made merely for the purpose of providing a full and enabling disclosure of the invention. The foregoing disclosure is not intended to be construed to limit the invention or otherwise exclude any such other embodiments, adaptations, variations, modifications or equivalent arrangements, the invention being limited only by the claims appended hereto and the equivalents thereof.

1-31. (canceled)

32. A method, comprising:
(a) inserting a sterilized textile item through an open end of a bag into an interior space of the bag and closing the open end of the bag such that the bag completely contains the item within the interior space, the bag comprising a sterilized textile material defining the interior space of the bag;
(b) folding a sheet of a sterilized textile material about the bag into a configuration such that the folded sheet of textile material defines an envelope that entirely envelops the bag; and
(c) securing the folded sheet of material in the configuration enveloping of the bag using an elongate length of a sterilized textile material.

33. The method of claim 32, further comprising before said steps (a), (b), and (c), sterilizing the textile item, sterilizing the sheet to be folded to define the envelope, and sterilizing the tie by washing, heating, or washing and heating.

34. The method of claim 33, wherein the sterilizing of the textile item, the sterilizing of the sheet, and the sterilizing of the tie are performed in the same process.

35. The method of claim 33, wherein the sterilizing of the textile item, the sterilizing of the sheet, and the sterilizing of the tie are performed in different processes.

36. The method of claim 32, further comprising after said steps (a), (b), and (c), and after the textile item has been unpacked and used, sterilizing the textile item, sterilizing the sheet to be folded to define the envelope, and sterilizing the tie by washing, heating, or washing and heating.

37. The method of claim 36, wherein the sterilizing of the textile item, the sterilizing of the sheet, and the sterilizing of the tie are performed in the same process.

38. The method of claim 36, wherein the sterilizing of the textile item, the sterilizing of the sheet, and the sterilizing of the tie are performed in different processes.

39. The method of claim 32, further comprising repackaging the textile item, 39, comprising:
(a) inserting the textile item through an open end of a bag into an interior space of the bag and closing the open end of the bag such that the bag completely contains the item within the interior space, the bag comprising a textile material;
(b) folding a sheet of a textile material about the bag into a configuration such that the folded sheet of textile material defines an envelope that entirely envelops the bag; and
(c) securing the folded sheet of material in the configuration enveloping of the bag.

40. The method of claim 32, wherein the sterilized, reusable textile item comprises a textile article used in foodservice.

41. The method of claim 40, wherein the textile article comprises an apron.

42. The method of claim 40, wherein the textile article comprises a head covering.

43. The method of claim 32, wherein the sterilized, reusable textile item comprises a textile article used in healthcare.

44. The method of claim 43, wherein the textile article comprises a gown.

45. The method of claim 43, wherein the textile article comprising a surgical drape.

46. The method of claim 43, wherein the textile article comprises scrubs.

47. The method of claim 43, wherein the textile article comprises a sanitary piece of textile clothing worn by a healthcare provider.

48. The method of claim 43, wherein the textile article comprises a sanitary piece of textile clothing worn by a healthcare patient.
